(19) United States
(12) Patent Application Publication
(10) Pub. No.: US 2024/0400981 A1
(43) Pub. Date: Dec. 5, 2024

(54) CELL POPULATION AND CELL POPULATION PRODUCTION METHOD

(71) Applicant: CellFiber Co., Ltd., Koto-ku, Tokyo (JP)

(72) Inventors: Kazuhiro IKEDA, Koto-ku, Tokyo (JP); Yuki TAKAGI, Koto-ku, Tokyo (JP); Lucas Siqueira TRINDADE, Koto-ku, Tokyo (JP)

(73) Assignee: CellFiber Co., Ltd., Koto-ku, Tokyo (JP)

(21) Appl. No.: 18/696,122

(22) PCT Filed: Jul. 7, 2022
(86) PCT No.: PCT/JP2022/027036

§ 371 Date: Mar. 27, 2024

(30) Foreign Application Priority Data
Sep. 30, 2021 (JP) 2021-162428

(51) Int. Cl. C12N 5/073

(52) U.S. Cl. CPC C12N 5/0605; C12N 2509/00; C12N 2513/00; C12N 2533/74; C12N 2537/10

(57) ABSTRACT

A cell population comprises a large number of cell aggregations and a hydrogel encapsulating the large number of cell aggregations. A coefficient of variation obtained by dividing a standard deviation of sizes of the large number of cell aggregations by a mean value of the sizes of the large number of cell masses is 0.50 or less. Alternatively and/or in addition there to, a coefficient of variation obtained by dividing a standard deviation of aspect ratios of shapes of the large number of cell aggregations by a mean value of the aspect ratios of the shapes of the large number of cell aggregations is 0.30 or less.

### TECHNICAL FIELD

The present invention relates to a cell population including a large number of cell aggregations, and a method for producing the cell population.

### BACKGROUND ART

Attempts have recently been made to culture various cells in a variety of fields such as developmental biology, drug discovery, and regenerative medicine. These cells are typically cultured two-dimensionally on the surface of a plastic dish for a tissue culture. There is known a technique for culturing cells in a three-dimensional or pseudo-three-dimensional culture environment using a scaffold such as a porous membrane and a hydrogel. Patent Literatures 1 to 6 below disclose that cells are cultured inside a tubular hydrogel.

In recent years, spheroid culture in which cells are cultured and aggregated three-dimensionally has attracted attention instead of monolayer culture in which cells are cultured two-dimensionally. The spheroid culture is known to be able to construct a state closer to cells in a living body as compared with the monolayer culture, and to be able to develop a specific function of the cells in the living body. Therefore, for example, in drug discovery research and the like, a cell aggregation is expected as a useful tool.

### CITATION LIST

### Patent Literature

Patent Literature 1: WO 2011/046105 A
Patent Literature 2: JP 2017-99303 A
Patent Literature 3: WO 2017/091662 A
Patent Literature 4: WO 2018/098295 A
Patent Literature 5: WO 2019/178549 A
Patent Literature 6: WO 2020/032221 A

### SUMMARY

Cell aggregations are desirably uniform in size, shape, and the like. Cell aggregations having a uniform size and shape are easy to handle in a similar manner and easily express an equivalent function. For example, in a case where cell aggregations are utilized for non-clinical trials of drug discovery research, a cell population including cell aggregations having a uniform size and/or shape is important in obtaining reliable test results.

In addition, it has been reported that a cell aggregation containing stem cells having differentiation potency changes in the direction of differentiation depending on its size. The cell population including cell aggregations having a uniform size and/or shape is important from such a viewpoint as well.

Therefore, a demand exists for a cell population including a large number of cell aggregations having a uniform size and/or shape, and a method for producing the cell population.

In aspect 1, a cell population comprising: a large number of cell aggregations; and a hydrogel encapsulating the large number of cell aggregations, wherein when a cell and a cell aggregation having a size less than a predetermined cut-off value is removed, a coefficient of variation obtained by dividing a standard deviation of sizes of the large number of cell aggregations by a mean value of the sizes of the large number of cell masses is 0.50 or less, and/or a coefficient of variation obtained by dividing a standard deviation of aspect ratios of shapes of the large number of cell aggregations by a mean value of the aspect ratios of the shapes of the large number of cell aggregations is 0.30 or less.

In aspect 2, the cell population according to the aspect 1, comprising: a tubular shell formed by the hydrogel; and a core provided inside the shell, the core including the large number of cell aggregations.

In aspect 3, the cell population according to the aspect 1, wherein the hydrogel has a capsule shape which is capable of dividing the large number of cell aggregations into at least one or more cell aggregations.

In aspect 4, the cell population according to any one of the aspects 1 to 3, wherein a storage modulus of the hydrogel in a state of encapsulating the large number of cell aggregations is 100 Pa or more.

In aspect 5, the cell population according to any one of the aspects 1 to 4, wherein the hydrogel is an alginate gel, and an M/G ratio of an alginic acid derivative for forming the alginate gel is 1.0 or less.

In aspect 6, the cell population according to any one of the aspects 1 to 5, wherein the hydrogel is an alginate gel, and an M/G ratio of an alginic acid derivative for forming the alginate gel is 0.1 or more. In aspect 7, the cell population according to the aspects 5 or 6, wherein the alginate gel is formed by gelling a solution in which a weight of the alginic acid derivative relative to a weight of a solvent containing the alginic acid derivative is between 0.5 wt % and 7.0 wt % inclusive.

In aspect 8, the cell population according to any one of the aspects 1 to 7, wherein a viscosity of a hydrogel derivative for forming the hydrogel is between 50 mPa and 10,000 mPa inclusive at a temperature of 20° C.

In aspect 9, a cell population comprising a large number of cell aggregations, wherein
when a cell and a cell aggregation having a size less than a predetermined cut-off value is removed,
   a coefficient of variation obtained by dividing a standard deviation of sizes of the large number of cell aggregations by a mean value of the sizes of the large number of cell aggregations is 0.50 or less,
   and/or a coefficient of variation obtained by dividing a standard deviation of aspect ratios of shapes of the large number of cell aggregations by a mean value of the aspect ratios of the shapes of the large number of cell aggregations is 0.30 or less.

In aspect 10, the cell population according to any one of the aspects 1 to 9, wherein the mean value of the aspect ratios of the shapes of the large number of cell aggregations is 1.5 or less when a cell and a cell aggregation having a size less than a predetermined cut-off value is removed.

In aspect 11, the cell population according to any one of the aspects 1 to 10, wherein the large number of cell aggregations contain adherent cells or non-adherent cells.

In aspect 12, the cell population according to any one of the aspects 1 to 11, wherein the large number of cell aggregations contain adherent cells that do not substantially proliferate in suspension.

In aspect 13, the cell population according to any one of the aspects 1 to 12, wherein each of the large number of cell aggregations contain different types of cells from each other.

In aspect 14, the cell population according to any one of the aspects 1 to 13, wherein the cell aggregations contain stem cell having multipotency or totipotency, progenitor cell having oligopotency or unipotency, or cell differentiated from stem cells or progenitor cells.

In aspect 15, the cell population according to any one of the aspects 1 to 14, wherein the cell aggregation contains cells differentiated from stem cells or progenitor cells in a state of being encapsulated in the hydrogel.

In aspect 16, a cell population comprising a large number of cell aggregations collected by removing the hydrogel constituting the cell population according to any one of the aspects 1 to 8.

In aspect 17, a method for producing a cell population comprising: encapsulating cells in a shell formed by a hydrogel; and culturing the cells in the shell so that, after removing a cell and a cell aggregation having a size less than a predetermined cut-off value, a coefficient of variation obtained by dividing a standard deviation of sizes of a large number of cell aggregations by a mean value of the sizes of the large number of cell aggregations is 0.50 or less, and/or a coefficient of variation obtained by dividing a standard deviation of aspect ratios of shapes of a large number of cell aggregations by a mean value of the aspect ratios of the shapes of the large number of cell aggregations is 0.30 or less.

In aspect 18, a method for producing a cell population comprising: encapsulating cells into a shell formed by a hydrogel; culturing the cells into the shell to form a large number of cell aggregations; and collecting the large number of cell aggregations from inside the shell to obtain a cell population in which after removing a cell and a cell aggregation having a size less than a predetermined cut-off value, a coefficient of variation obtained by dividing a standard deviation of sizes of a large number of cell aggregations by a mean value of the sizes of the large number of cell aggregations is 0.50 or less, and/or a coefficient of variation obtained by dividing a standard deviation of aspect ratios of shapes of a large number of cell aggregations by a mean value of the aspect ratios of the shapes of the large number of cell aggregations is 0.30 or less.

In aspect 19, the method for producing a cell population according to the aspect 18, wherein the large number of cell aggregations are collected from inside the shell by dissolving the hydrogel solution using a solution containing EDTA or an alginate lyase.

In aspect 20, the method for producing a cell population according to any one of the aspects 17 to 19, wherein the cell aggregation contain stem cell having multipotency or totipotency, and/or progenitor cell having oligopotency or unipotency, the method comprising differentiating the stem cells and/or the progenitor cells in a state of being encapsulated in the hydrogel.

In aspect 21, a biological substance generated by the large number of cell aggregations constituting the cell population according to any one of the aspects 1 to 16.

According to the above aspects, it is possible to provide a cell population including cell aggregations having a uniform size and/or shape.

### BRIEF DESCRIPTION OF DRAWINGS

FIG. 1 is a micrograph showing an example of a cell population including a large number of cell aggregations.

FIG. 2 is a schematic view illustrating the cross section of a tubular hydrogel.

FIG. 3 is a schematic view illustrating an example of a device for producing a cell population.

FIG. 4 is an enlarged micrograph of a cell population according to Example 22.

FIG. 5 is a photograph showing a state of the cell population according to Example 22 observed with a fluorescence microscope, in which only human iPS cell-derived cardiomyocytes emit fluorescence.

FIG. 6 is a micrograph of cell aggregations constituting a cell population in Example 32 on day 1 of culture.

FIG. 7 is a micrograph of the cell aggregations constituting the cell population in Example 32 on day 7 of culture.

FIG. 8 is a micrograph of differentiated cells in Example 31.

### DESCRIPTION OF EMBODIMENTS

As a result of intensive studies, the present inventors have found a cell population including a large number of cell aggregations having a uniform size and/or shape, and a method for producing the cell population.

### [Cell Population Including a Large Number of Cell Aggregations Encapsulated in Hydrogel]

A cell population may include a large number of cell aggregations and a hydrogel encapsulating the large number of cell aggregations. In this case, the cell population is defined by a large number of cell aggregations encapsulated in one or more hydrogels cultured under the same conditions. More preferably, the cell population is defined by a large number of cell aggregations encapsulated in one hydrogel.

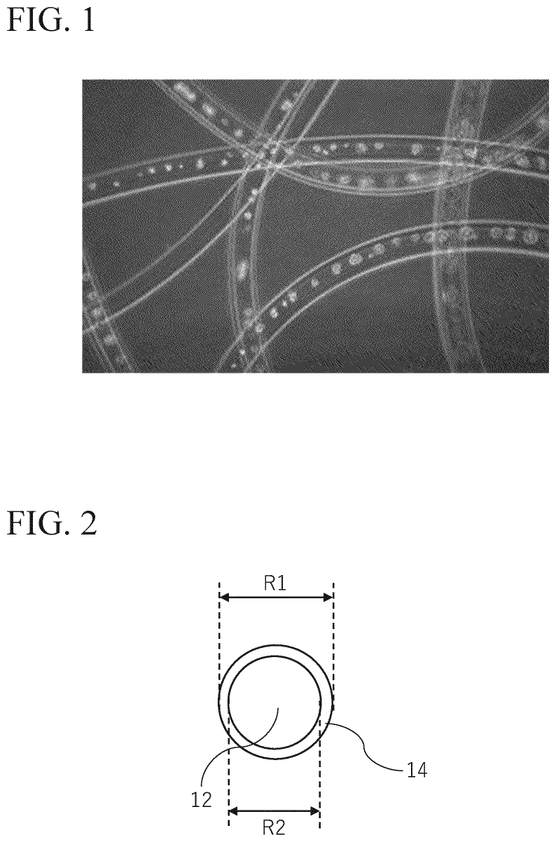

FIG. 1 is a micrograph showing an example of the cell population including the large number of cell aggregations. FIG. 1 shows a state in which the large number of cell aggregations are encapsulated in a tubular hydrogel. FIG. 2 is a schematic view illustrating the cross section of the tubular hydrogel.

As illustrated in FIGS. 1 and 2, the hydrogel may have a tubular shape. More specifically, the cell population may include a tubular shell 14 formed by the hydrogel, and a core 12 provided in the shell 14. The core 12 may include the large number of cell aggregations.

The core 12 may extend long in a string shape. The shell 14 may surround the periphery of the core 12 and extend along the string-like extending core 12.

The length of the hydrogel may be, for example, 5 cm or more, more preferably 10 cm or more, and still more preferably 20 cm or more. The longer the length of the hydrogel, the more cell aggregations can be produced in one hydrogel. This makes it easy to increase the number of cell aggregations belonging to the cell population.

The cell aggregations constituting the cell population may contain adherent cells or non-adherent cells. Each cell aggregation may be a cluster formed only by adherent cells, may be a cluster formed only by non-adherent cells, or may be a cluster formed by both the adherent cells and the non-adherent cells. The adherent cells may be, for example, cells that do not substantially proliferate in suspension.

The adherent cells are not limited to a particular type. The adherent cells may be various stem cells having multipotency, human ES cells or human iPS cells having pluripotency, stem cells having multipotency, progenitor cells having oligopotency or unipotency, or the like. Examples of various stem cells having multipotency and totipotency include iPS cells, ES cells, mesenchymal stem cells, neural stem cells, hepatic stem cells, skin stem cells, and pancreatic stem cells. Examples of the stem cells and the progenitor cells having oligopotency or unity include muscle stem cells, germ stem cells, respiratory progenitor cells, intestinal progenitor cells, and cardiac progenitor cells. In addition, the adherent cells may be various differentiated cells, e.g., muscle cells such as skeletal muscle cells, smooth muscle cells, and cardiomyocytes, nerve cells such as cerebral cortex cells, fibroblasts, epithelial cells, endothelial cells, vascular endothelial cells, adipocytes, osteoblasts, chondrocytes, macrophages, dendritic cells, hepatocytes, hepatic stellate cells, pancreatic B cells, keratinocytes, nephrocytes, kidney tubular cells, hair matrix cells, corneal endothelial cells, photoreceptor cells, pigment epithelial cells, goblet cells, respiratory cells, or the like. These cells may be cells produced by inducing differentiation from cells having pluripotency or multipotency.

For example, mesenchymal stem cells, muscle cells, hepatic stellate cells, and fibroblasts are cells having relatively high adhesiveness. Various differentiated cells and the other cells described above are cells having relatively low adhesiveness.

The cell aggregations constituting the cell population may contain cells that are originally non-adherent but have adhesiveness by being subjected to proliferative stimulation. Such cells may be, for example, blood cells, T cells, NK cells, lymphoid progenitor cells, hematopoietic stem cells, suspension CHO cells/suspension HEK 293, or the like.

The inventors of the present application have found that by culturing such adherent cells in the tubular hydrogel, the sizes and aspect ratios of the cell aggregations can be uniformly controlled as described later.

The non-adherent cells are not limited to a particular type. The non-adherent cells may be, for example, blood cells (such as K 562), T cells, NK cells, lymphoid progenitor cells, or the like. In addition, the non-adherent cells may be those obtained by suspension-culturing and acclimating an adherent cell line. Examples of such non-adherent cells include CHO cells and HEK 293.

The inventors of the present application have found that by culturing such non-adherent cells in the tubular hydrogel, even the non-adherent cells can form a cell aggregation. Furthermore, the inventors of the present application have found that the shapes, for example, the sizes of the cell aggregations formed by the non-adherent cells can be uniformly controlled as described later.

Each of the cell aggregations constituting the cell population may be formed by only one type of cell or may be formed by different types of cells from each other. In a case where the cell aggregation contains different types of cells from each other, only one type of the cells forming each cell aggregation may be adherent cells or non-adherent cells. Preferably, all of the plurality of types of cells forming the cell aggregation may be adherent cells or non-adherent cells.

In this case, a combination of the cell types constituting the cell aggregation may be any combination of the cell types described above. The combination of different types of cells constituting the cell aggregation may be, for example, any combination selected from various stem cells having multipotency, totipotency, or unity of differentiation, progenitor cells derived from these stem cells, and cells differentiated from these stem cells. The cells thus differentiated may be, for example, vascular endothelial cells, fibroblasts, or the like. In addition, the combination of the cell types constituting the cell aggregation may be a combination of myocardial, hepatic, respiratory, skin, pancreatic, or intestinal stem cells, and progenitor cells thereof.

As described above, the cell type constituting the cell aggregation may include stem cells having multipotency or totipotency, progenitor cells having oligopotency or unipotency, or cells differentiated from the stem cells or the progenitor cells. In a case where the cell aggregation contains stem cells or progenitor cells, the stem cells or the progenitor cells can be differentiated in a state of being encapsulated in the hydrogel.

In a case where the cell aggregation contains cells differentiated from the stem cells or the progenitor cells, the cell aggregation may contain cells differentiated from the stem cells or the progenitor cells in a state of being encapsulated in the hydrogel. However, it should be noted that cells differentiated by a normal process may be encapsulated in the hydrogel and then cultured.

In a case where the stem cells or the progenitor cells are differentiated in the state of being encapsulated in the hydrogel, these cells may be, for example, iPS cells or mesenchymal stem cells. In particular, the mesenchymal stem cells have an advantage of being easily differentiated under a size condition suitable for differentiation because the size of the mesenchymal stem cells is easily controlled for application in the state of being encapsulated in the hydrogel. Furthermore, a small variation in the size and/or aspect ratio of the cell aggregation before differentiation is expected to increase the proportion of the cell aggregation to be differentiated as intended. In particular, since it is known that the size and the like of the cell aggregation containing the mesenchymal stem cells affect the differentiation potency, there is a possibility that the mesenchymal stem cells can be efficiently differentiated by being differentiated in the state of being encapsulated in the hydrogel.

In a case where the stem cells or the progenitor cells are differentiated in the state of being encapsulated in the hydrogel, the stem cells or the progenitor cells only have to be immersed and cultured in a differentiation induction medium together with the hydrogel. Differentiation of cells in the state of being encapsulated in the hydrogel can be performed in the same manner as in a normal differentiation method except for the use of the hydrogel.

The cells after differentiation in the state of being encapsulated in the hydrogel may be, for example, chondrocytes, adipocytes, osteoblasts, nerve cells, muscle cells, fibroblasts, hepatocytes, islet cells or islet cell-like cells, or the like.

The core may include, for example, at least one selected from the group consisting of a physiological saline solution, an aqueous solution of an inorganic salt, an aqueous solution of saccharides, a culture medium, a culture supernatant, a buffer solution, an extracellular matrix, a thickener, and a hydrophilic resin (polyvinyl alcohol, polyacrylamide, poly N-isopropyl acrylamide, and the like).

The thickener may include, for example, at least one selected from the group consisting of chitosan gel, collagen solution, matrigel, collagen, gelatin, alginic acid solution, alginate gel, peptide gel, laminin, agarose, nanocellulose, methylcellulose, hyaluronic acid, proteoglycan, elastin, pullulan, dextran, pectin, gellan gum, xanthan gum, guar gum, carrageenan, and glucomannan. As will be described later, from the viewpoint of uniformizing the aspect ratios of the shapes of a large number of cell aggregations, the core preferably includes the thickener.

The core may include various growth factors suitable for cell culture, cell maintenance and proliferation, cell functional expression, or the like. Such growth factors may be, for example, at least one selected from the group consisting of an epidermal growth factor (EGF), a platelet-derived growth factor (PDGF), a transforming growth factor (TGF), an insulin-like growth factor (IGF), a fibroblast growth factor (FGF), a nerve growth factor (NGF), a vascular endothelial growth factor (VEGF), and a hepatocyte growth factor (HGF).

The hydrogel is obtained by gelling a hydrogel precursor. The hydrogel can also function as a scaffold for adherent cells. In this case, the hydrogel preferably has a sufficient strength as the scaffold for adherent cells. More preferably, the hydrogel can have sufficient permeability to cell culture medium components.

On the other hand, the hydrogel may not function as the scaffold. In this case, the hydrogel may have sufficient permeability to cell culture medium components while confining the cells. For example, in a case where the hydrogel is an alginate gel, the above-described adherent cells can be cultured without adhering to the alginate gel. In this case, the adherent cells are easily aggregated together in a space inside the hydrogel to form a substantially spherical spheroid without adhering to the wall of the alginate gel. From this viewpoint, in a preferred example, the cells include mesenchymal stem cells and the hydrogel includes an alginate gel.

The hydrogel may include, for example, at least one selected from the group consisting of alginate gel, matrigel, collagen gel, chitosan gel, gelatin, peptide gel, laminin gel, agarose gel, nanocellulose, methylcellulose, xtran, pectin, gellan gum, xanthan gum, guar gum, carrageenan, glucomannan, fibrin gel, and tetra-peg gel.

Preferably, the hydrogel forming the shell is a gel containing an alginate gel as a main component, i.e. an alginate gel. In this case, the hydrogel precursor may be a solution containing an alginic acid solution as a main component.

The alginate gel can be formed by gelling an alginic acid derivative (alginic acid precursor). The alginic acid derivative may be an alginic acid solution. The alginate gel can be formed by crosslinking an alginic acid solution as the alginic acid derivative with divalent metal ions. The alginic acid solution may be, for example, sodium alginate, potassium alginate, or ammonium alginate, or a combination thereof. The alginic acid solution is easily crosslinked with divalent metal ions in a short time at normal temperature or around normal temperature to easily become an alginate gel. In addition, the alginate gel has low cytotoxicity. Therefore, the alginate gel can be suitably used as the hydrogel for culturing cells or cell aggregations in a state of encapsulating the cells or cell aggregations.

The alginic acid derivative may be a natural extract or may be chemically modified. Examples of the chemically-modified alginic acid include methacrylate-modified alginic acid. In addition, the hydrogel may be a mixed system of the alginate described above and collagen, gelatin, agar, agarose, polyethylene glycol (PEG), polylactic acid (PLA), nanocellulose, or the like.

The divalent metal ions used for crosslinking the alginic acid derivative may be, for example, calcium ions, magnesium ions, barium ions, strontium ions, zinc ions, iron ions, or the like. Preferably, the divalent metal ions are calcium ions or barium ions.

The divalent metal ions are preferably provided to the alginic acid in a solution form. Examples of a solution containing the divalent metal ions include a solution containing calcium ions. Examples of such a solution include an aqueous solution such as an aqueous calcium chloride solution, an aqueous calcium carbonate solution, an aqueous calcium gluconate solution, and an aqueous calcium lactate solution. Preferably, such a solution may be an aqueous calcium chloride solution or an aqueous barium chloride solution.

The concentration of the divalent metal ions in the solution containing the divalent metal ions is, for example, 1 mM to 1 M, preferably 20 to 500 mM, and more preferably 100 mM.

The outer diameter of the hydrogel forming the tubular shell (see “R1” in FIG. 2) is not particularly limited, but may be, for example, in a range of 40 μm to 4,000 μm, preferably in a range of 80 μm to 2,000 μm, and more preferably in a range of 100 μm to 1,000 μm. Herein, the outer diameter of the shell may be defined by a mean value of outer diameters measured at a plurality of positions, for example, 10 positions.

The inner diameter of the hydrogel forming the tubular shell (see “R2” in FIG. 2) only has to be determined from the viewpoint of controlling a mean value of the sizes of the large number of cell aggregations. The inner diameter of the hydrogel is not particularly limited, but may be, for example, in a range of 10 μm to 3,000 μm, preferably in a range of 20 μm to 1,000 μm, and more preferably in a range of 40 μm to 500 μm. Herein, the inner diameter of the shell may be defined by a mean value of inner diameters measured at a plurality of positions, for example, 10 positions. The inner diameter and the outer diameter described above can be measured by, for example, a phase contrast optical microscope.

The inner diameter of the hydrogel can limit the upper limit of the mean value of the sizes of the large number of cell aggregations encapsulated in the hydrogel. Therefore, the inner diameter of the hydrogel only has to be determined so that the mean value of the sizes of the large number of cell aggregations approaches a desired value. The mean value of the sizes of the large number of cell aggregations constituting the cell population is not particularly limited, but may be, for example, in a range of 40 μm to 500 μm.

In the present specification, it should be noted that the mean value of the sizes of the large number of cell aggregations constituting the cell population is a value calculated under a condition of removing cells and cell aggregations having a size less than a predetermined cut-off value. The same cut-off value is similarly used in calculating the size or the aspect ratio described later, its mean value, its standard deviation, and the like. Herein, the cut-off value may be 30 μm from the viewpoint of removing cells or cell aggregations that absolutely do not form a cell aggregation, or scarcely form a cell aggregation.

From the viewpoint of reducing a variation in the sizes of the large number of cell aggregations constituting the cell population, a coefficient of variation obtained by dividing the standard deviation of the sizes of the large number of cell aggregations encapsulated in the hydrogel by the mean value of the sizes of the large number of cell aggregations after removing the cells and cell aggregations having a size less than the predetermined cut-off value is, for example, 0.50 or less, preferably 0.40 or less, more preferably 0.35 or less, and still more preferably 0.30 or less. In this aspect, it should be noted that by culturing cells or cell aggregations inside the hydrogel as described below, a cell population having a small variation in the sizes of such cell aggregations can be provided.

The sizes of the large number of cell aggregations, the mean value and the standard deviation thereof, and the coefficient of variation may be calculated using, as a population, a large number of cell aggregations encapsulated in one or more hydrogels formed and cultured under the same conditions. Thus, the cell population is constituted by the large number of cell aggregations encapsulated in one or more hydrogels formed and cultured under the same conditions. A large number of cell aggregations cultured under the same conditions in a plurality of hydrogels (shells) formed under the same conditions may be treated as belonging to the same cell population.

Preferably, the sizes of the large number of cell aggregations, the mean value and the standard deviation thereof, and the coefficient of variation may be calculated using, as a population, a large number of cell aggregations encapsulated in one tubular hydrogel. In this case, the cell population is calculated using the large number of cell aggregations encapsulated in one tubular hydrogel as a population. In this case, the cell population is constituted by the large number of cell aggregations encapsulated in one hydrogel.

In the present specification, in the state where the large number of cell aggregations are encapsulated in the tubular hydrogel, the size (thickness) of each cell aggregation described above may be defined by the length of the cell aggregation in a radial direction of the tubular hydrogel.

Specifically, the size of the cell aggregation may be defined by the length in the radial direction of the hydrogel in a two-dimensional plane of the cell aggregation projected on a two-dimensional projection surface such as a micrograph. It should be noted that the size of the cell aggregation does not necessarily have to be defined by the maximum or minimum length in a three-dimensional shape of the aggregations. In a state in which the large number of cell aggregations are not encapsulated in the tubular hydrogel, the size of each cell aggregation described above may be defined by the length in a minor axis direction of the cell aggregation. Herein, the mean value, the standard deviation, and the like of the sizes of the large number of cell aggregations constituting the cell population are calculated under a condition of removing cells and cell aggregations having a size in which the length of the cell aggregation in the radial direction of the tubular hydrogel is less than the above-described cut-off value.

The lower limit of the coefficient of variation obtained by dividing the standard deviation of the sizes of the large number of cell aggregations encapsulated in the hydrogel by the mean value of the sizes of the large number of cell aggregations is not particularly limited. The coefficient of variation obtained by dividing the standard deviation of the sizes of the large number of cell aggregations encapsulated in the hydrogel by the mean value of the sizes of the large number of cell aggregations after removing the cells and cell aggregations having a size less than the predetermined cut-off value may be, for example, 0.10 or more, and in another example, 0.12 or more.

It should be noted that in a case where the cells are differentiated in the state of being encapsulated in the hydrogel, the sizes of the cell aggregations before differentiation, the standard deviation of the sizes, the coefficient of variation thereof, and the like may satisfy the above values, and the sizes of the cell aggregations after differentiation, the standard deviation of the sizes, the coefficient of variation thereof, and the like may satisfy the above values.

Instead of the value of the coefficient of variation related to the sizes of the cell aggregations described above or in addition to the value of this coefficient of variation, a coefficient of variation obtained by dividing the standard deviation of the aspect ratios of the shapes of the large number of cell aggregations encapsulated in the hydrogel by the mean value of the aspect ratios of the shapes of the large number of cell aggregations after removing the cells and cell aggregations having a size less than the predetermined cut-off value is, for example, 0.30 or less, preferably 0.28 or less, and more preferably 0.26 or less. This means that the shapes of the large number of cell aggregations constituting the cell population are uniformized. In this aspect, it should be noted that by culturing cells or cell aggregations inside the hydrogel as described below, a cell population having a small variation in the aspect ratios of the shapes of such cell aggregations can be provided.

The lower limit of the coefficient of variation obtained by dividing the standard deviation of the aspect ratios of the shapes of the large number of cell aggregations encapsulated in the hydrogel by the mean value of the aspect ratios of the shapes of the large number of cell aggregations is not particularly limited. The coefficient of variation obtained by dividing the standard deviation of the aspect ratios of the shapes of the large number of cell aggregations encapsulated in the hydrogel by the mean value of the aspect ratios of the shapes of the large number of cell aggregations after removing the cells and cell aggregations having a size less than the predetermined cut-off value may be, for example, 0.06 or more, and in another example, 0.08 or more.

Herein, the aspect ratio of the shape of each cell aggregation is defined by a ratio between the length in a major axis direction and the length in a minor axis direction of an elliptical shape when the cell aggregation is regarded as having an elliptical shape in a state of being projected on a two-dimensional projection surface such as a micrograph, that is, a value obtained by dividing the length in the major axis direction by the length in the minor axis direction.

The mean value of the aspect ratios of the shapes of the large number of cell aggregations constituting the cell population after removing the cells and cell aggregations having a size less than the predetermined cut-off value may be, for example, 2.00 or less, preferably 1.50 or less, more preferably 1.35 or less, and still more preferably 1.30 or less. This means that the large number of cell aggregations constituting the cell population have a nearly spherical shape.

The lower limit of the mean value of the aspect ratios of the shapes of the large number of cell aggregations constituting the cell population is not particularly limited. The mean value of the aspect ratios of the shapes of the large number of cell aggregations constituting the cell population after removing the cells and cell aggregations having a size less than the predetermined cut-off value may be, for example, 1.00 or more, 1.10 or more in another example, and 1.14 or more in still another example.

It should be noted that in a case where the cells are differentiated in the state of being encapsulated in the hydrogel, the aspect ratios of the shapes of the large number of cell aggregations before differentiation, the standard deviation of the aspect ratios, the coefficient of variation thereof, and the like may satisfy the above values, and the aspect ratios of the cell aggregations after differentiation, the standard deviation of the aspect ratios, the coefficient of variation thereof, and the like may satisfy the above values.

Herein, in a case where the cells are cultured in the tubular hydrogel, the cells may have a shape extending along an extending direction of the tubular hydrogel. The present inventors have found that the large number of cell aggregations constituting the cell population can achieve a cell population having a small mean value of the aspect ratios as described above depending on conditions even when cultured in the tubular hydrogel.

In a case where the hydrogel is an alginate gel, an M/G ratio of the alginic acid derivative may be appropriately set. The M/G ratio corresponds to the ratio of mannuronic acid (M) to gluronic acid (G) contained in the alginic acid derivative. The M/G ratio of the alginic acid derivative affects gelling ability and gel strength. It is known that in a case where the M/G ratio is relatively low, that is, the proportion of gluronic acid (G) is relatively high, the gel strength of the alginate gel obtained by crosslinking the alginic acid derivative increases.

When the gel strength of the alginate gel is high, it is considered that the cell aggregations in the shell are cultured in a space restricted by the shell. In general, when adherent cells are subjected to suspension culture in a predetermined space or plane, aggregates having a uniform size are once formed. However, since the cell aggregations adhere to or are fused to each other by slight vibration or replacement of solutions, it is difficult to control the sizes and aspect ratios of the cell aggregations. On the other hand, the influence of motion of a liquid outside the hydrogel is blocked inside the hydrogel, so that adhesion and fusion between the cell aggregations hardly occur, and the sizes of the cell aggregations once formed can be maintained relatively uniformly. Furthermore, even in a case where the cells initially introduced into the hydrogel are dense, the sizes of the cell aggregations in the shell are controlled to some extent by the size (for example, the inner diameter) of the shell, so that the sizes of the large number of cell aggregations easily become uniform. From this viewpoint, the M/G ratio of the alginic acid derivative may be, for example, 1.0 or less, preferably 0.8 or less, and more preferably 0.6 or less.

The molecular weight of the alginic acid derivative, e.g., sodium alginate may be, for example, 60,000 or more, and preferably 100,000 or more. It is known that the gel strength increases as the molecular weight of the alginic acid derivative is higher. As the gel strength of the alginate gel is higher, the influence of motion of a liquid outside the gel is blocked as described above, so that adhesion and fusion between the cell aggregations hardly occur. Therefore, from the viewpoint of making the sizes of the large number of cell aggregations uniform, it is preferable that the molecular weight of the alginic acid derivative is high as described above.

The molecular weight of the alginic acid derivative, e.g., sodium alginate, may be, for example, 3,000,000 or less, and in another example, 2,000,000 or less.

Herein, the above-described molecular weight may be a weight average molecular weight measured by gel permeation chromatography (GPC) or gel filtration chromatography.

In addition, it is considered that the higher the concentration of the alginic acid derivative for generating the alginate gel, the higher the gel strength. Therefore, from the viewpoint of making the sizes of the large number of cell aggregations in the alginate gel uniform, the concentration of the alginic acid derivative, that is, the weight of the alginic acid derivative relative to the weight of a solvent containing the alginic acid derivative may be, for example, 7.0 wt % or less, preferably 5.0 wt % or less, and more preferably 3.0 wt % or less. In particular, in a case where the concentration of the alginic acid derivative falls within this range and the M/G ratio of the alginate gel and/or the molecular weight of the alginate gel fall(s) within the above-described range(s), the viscosity and/or gel strength of the alginate gel derivative easily have (has) an appropriate value from the viewpoint of reducing the variation in the sizes and aspect ratios of the cell aggregations.

In addition, from the viewpoint of reducing a cell leakage from the inside of the hydrogel, the concentration of the alginic acid derivative, that is, the weight of the alginic acid derivative relative to the weight of the solvent containing the alginic acid derivative may be, for example, 0.5 wt % or more, and preferably 0.75 wt % or more.

A storage modulus (G′) at a frequency of 1 Hz of the hydrogel forming the shell in the state of encapsulating the large number of cell aggregations may be 100 Pa or more, preferably 180 Pa or more, and more preferably 400 Pa or more. The value of the storage modulus (G′) may be a value measured at a temperature of 28° C. It is considered that the hydrogel having such a storage modulus has a relatively high gel strength and easily reduces the variation in the sizes of the large number of cell aggregations.

It is considered that when the gel strength of the alginate gel is low, the size of the shell increases along with an increase in the sizes of the cell aggregations. Thus, the sizes of the cell aggregations are believed to be less restricted by the initial size (e.g., the inner diameter) of the shell. In this case, it is considered that the cell aggregations in the shell relatively easily have a spherical shape. In other words, it is considered that the aspect ratios of the shapes of the cell aggregations easily become uniform and/or approach “1”. From this viewpoint, the M/G ratio of the alginic acid derivative is, for example, 0.05 or more, and preferably 0.1 or more.

In addition, it is considered that the lower the concentration of the alginic acid derivative for generating the alginate gel, the lower the gel strength. Therefore, from the viewpoint of the aspect ratios of the shapes of the large number of cell aggregations in the alginate gel, the weight of the alginic acid derivative relative to the weight of the solvent containing the alginic acid derivative is, for example, 0.2 wt % or more, preferably 0.5 wt % or more, and more preferably 0.8 wt % or more.

The storage modulus (G′) at a frequency of 1 Hz of the hydrogel forming the shell in the state of encapsulating the large number of cell aggregations may be 10,000 Pa or less, and preferably 8,000 Pa or more. The value of the storage modulus (G′) may be a value measured at a temperature of 28° C. The hydrogel having such a storage modulus is not too hard, and is considered to be suitable from the viewpoint of making the aspect ratios of the shapes of the cell aggregations uniform.

The viscosity of a hydrogel derivative such as the alginic acid derivative is not particularly limited, but may be, for example, 50 mPa·s or more, preferably 100 mPa·s or more, and more preferably 250 mPa·s or more at 20° C. As a result, the gel strength of the hydrogel becomes relatively high, making it easy to reduce the variation in the sizes of the cell aggregations that can be produced.

In addition, the viscosity of the hydrogel derivative such as the alginic acid derivative is not particularly limited, but may be, for example, 10,000 mPa·s or less, preferably 5,000 mPa's or less, and more preferably 1,000 mPa·s or less at 20° C. As a result, the gel strength of the hydrogel does not become too high, and more preferable cell culture conditions can be provided.

The cells may be cultured in a culture plate in the state of being encapsulated in the hydrogel, or may be cultured under suspension culture conditions. In any case, the cells are cultured with the hydrogel immersed in a culture medium.

The number of days of culture of the cells encapsulated in the hydrogel is not particularly limited, and only has to be appropriately set according to the cell type so that the variation in the sizes of the cell aggregations and the variation in the aspect ratios of the cell aggregations can be reduced.

The number of days of culture for obtaining the cell aggregations may be reduced as the adhesiveness of the cells encapsulated in the hydrogel increases. On the other hand, even in a case where the cell adhesiveness is low (including non-adhesiveness), cell aggregations having a small variation in the sizes and/or aspect ratios can be formed by increasing the number of days of culture. Typically, in the case of the above-described cells having relatively high adhesiveness, the number of days of culture can be between 0.5 days and 14 days inclusive, and specifically between 1 day and 7 days inclusive (preferably 4 days or less, 3 days or less, or 2 days or less). In the case of the above-described cells having relatively low adhesiveness, the number of days of culture can be between 3 days and 30 days inclusive, and specifically more than 7 days (preferably 10 days or more, 13 days or more, 15 days or more, 17 days or more, 19 days or more, or 21 days or more) and 25 days or less.

The number of days of culture for obtaining the cell aggregations may be reduced as the hydrogel becomes harder (typically satisfies the above-described M/G ratio and/or storage modulus). On the other hand, even in a case where the hydrogel is soft (typically, does not satisfy the above-described M/G ratio and/or storage modulus), cell aggregations having a small variation in the sizes and/or aspect ratios can be formed by increasing the number of days of culture.

In the above-described aspect, the tubular shell including the hydrogel extends long, and the core extends long in a string shape and is encapsulated in the tubular shell. That is, both the core and the shell extend substantially equally long. Alternatively, the hydrogel may have a capsule shape capable of dividing the large number of cell aggregations into at least one or more cell aggregations. For example, the hydrogel may have an elliptical or circular (substantially spherical) shape. In addition, a plurality of hydrogels having such a shape may be used. In this case, the sum of the large number of cell aggregations encapsulated in the plurality of hydrogels may constitute one cell population.

In addition, the hydrogel may be, for example, a plurality of elliptical, circular (substantially spherical), tubular, or string-like hydrogels bonded to each other. For example, in a case where a plurality of elliptical or circular (substantially spherical) hydrogels are bonded to each other, one or more cell aggregations are adjacent to each other with a partition wall of the hydrogel interposed therebetween. In this case, the hydrogels also have a capsule shape capable of dividing the large number of cell aggregations into at least one or more cell aggregations.

The sizes and aspect ratios of the cell aggregations can be controlled by the hydrogels in these cases as well, so that it is expected that the cell population having a uniform size and/or aspect ratio can be easily formed. However, from the viewpoint of facilitating collective handling of the large number of cell aggregations and/or securing sufficient permeability to cell culture medium components, it is preferable that the shapes of the core and the shell described above are substantially coaxial with each other and extend long as illustrated in FIGS. 1 and 2.

### [Cell Population Including a Large Number of Collected Cell Aggregations]

A cell population according to one aspect includes a large number of cell aggregations collected by removing the hydrogel encapsulating the large number of cell aggregations constituting the cell population described above. That is, the cell population may be defined by a large number of cell aggregations not including the hydrogel.

Thus, the cell population may include a large number of cell aggregations without substantially including the above-described hydrogel. In this case, the cell population may be defined by a large number of cell aggregations obtained by collecting the cell aggregations encapsulated in one or more hydrogels cultured under the same conditions. More preferably, the cell population is defined by a large number of cell aggregations obtained by collecting the cell aggregations encapsulated in one tubular hydrogel. In this way, the cell population may be defined by a large number of cell aggregations in a state of not being encapsulated in the hydrogel.

It can be predicted that the sizes and aspect ratios of the large number of cell aggregations collected from the hydrogel, the variation in these values, and the like greatly change from those values in the state of being encapsulated in the hydrogel. However, the inventors of the present application have found that, contrary to such prediction, the sizes and aspect ratios of the large number of cell aggregations collected from the hydrogel, the variation in these values, and the like do not significantly change in the large number of collected cell aggregations (cell population). It is considered that such findings regarding the size and the shape are established especially for a spheroid formed by the aggregating property of adherent cells. Therefore, the above-described numerical values and numerical ranges regarding the sizes and aspect ratios of the large number of cell aggregations constituting the cell population in the state of being encapsulated in the hydrogel can be substantially established for the large number of collected cell aggregations (cell population).

Therefore, the coefficient of variation obtained by dividing the standard deviation of the sizes of the large number of cell aggregations constituting the collected cell population by the mean value of the sizes of the large number of cell aggregations after removing the cells and cell aggregations having a size less than the predetermined cut-off value is, for example, 0.50 or less, preferably 0.40 or less, more preferably 0.35 or less, and still more preferably 0.30 or less.

The lower limit of the coefficient of variation obtained by dividing the standard deviation of the sizes of the large number of collected cell aggregations by the mean value of the sizes of the large number of cell aggregations is not particularly limited. The coefficient of variation obtained by dividing the standard deviation of the sizes of the large number of collected cell aggregations by the mean value of the sizes of the large number of cell aggregations after removing the cells and cell aggregations having a size less than the predetermined cut-off value may be, for example, 0.10 or more, and in another example, 0.12 or more.

In the present specification, the size of each cell aggregation not encapsulated in the hydrogel, for example, the size of each cell aggregation collected from the hydrogel is defined by the length in a minor axis direction of an elliptical shape when the cell aggregation is regarded as having an elliptical shape in a state of being projected on a two-dimensional projection surface such as a micrograph. In this case, it should be noted that the mean value, the standard deviation, and the like of the sizes or aspect ratios of the large number of cell aggregations constituting the cell population are calculated under a condition of removing cells and cell aggregations in which the length of the cell aggregation in the minor axis direction is less than the cut-off value.

Instead of the value of the coefficient of variation related to the sizes of the cell aggregations described above or in addition to the value of this coefficient of variation, the coefficient of variation obtained by dividing the standard deviation of the aspect ratios of the shapes of the large number of collected cell aggregations by the mean value of the aspect ratios of the shapes of the large number of cell aggregations after removing the cells and cell aggregations having a size less than the predetermined cut-off value is, for example, 0.30 or less, preferably 0.28 or less, and more preferably 0.26 or less.

The lower limit of the coefficient of variation obtained by dividing the standard deviation of the aspect ratios of the shapes of the large number of collected cell aggregations by the mean value of the aspect ratios of the shapes of the large number of cell aggregations is not particularly limited. The coefficient of variation obtained by dividing the standard deviation of the aspect ratios of the shapes of the large number of collected cell aggregations by the mean value of the aspect ratios of the shapes of the large number of cell aggregations after removing the cells and cell aggregations having a size less than the predetermined cut-off value may be, for example, 0.06 or more, and in another example, 0.08 or more.

The mean value of the aspect ratios of the shapes of the large number of collected cell aggregations after removing the cells and cell aggregations having a size less than the predetermined cut-off value may be, for example, 2.00 or less, preferably 1.50 or less, more preferably 1.35 or less, and still more preferably 1.30 or less.

The lower limit of the mean value of the aspect ratios of the shapes of the large number of collected cell aggregations is not particularly limited. The mean value of the aspect ratios of the shapes of the large number of collected cell aggregations after removing the cells and cell aggregations having a size less than the predetermined cut-off value may be, for example, 1.00 or more, 1.10 or more in another example, and 1.14 or more in still another example.

In the present specification, the aspect ratio of each cell aggregation not encapsulated in the hydrogel, for example, the aspect ratio of each cell aggregation collected from the hydrogel is defined by a ratio between the length in a major axis direction and the length in a minor axis direction of an elliptical shape when the cell aggregation is regarded as having an elliptical shape in a state of being projected on a two-dimensional projection surface such as a micrograph, that is, a value obtained by dividing the length in the major axis direction by the length in the minor axis direction.

### [Mean Value of Sizes of a Large Number of Cell Aggregations]

A method for calculating the mean value of the sizes of the large number of cell aggregations constituting the cell population will be described. First, the large number of cell aggregations constituting the cell population are micrographed. Herein, when the large number of cell aggregations are in the state of being encapsulated in the hydrogel, the micrograph only has to be obtained in the state of being encapsulated in the hydrogel.

The mean values of the sizes and aspect ratios of the large number of cell aggregations are calculated by values obtained by dividing the sums of the sizes and aspect ratios of the large number of cell aggregations by the number of large number of cell aggregations. Herein, the number of cell aggregations may be the total number of cell aggregations constituting the cell population. Alternatively, the number of cell aggregations may be the number of some cell aggregations (samples) constituting the cell population. In this case, it is considered that each of the mean values of the sizes and aspect ratios of the cell aggregations is an estimated value calculated using some cell aggregations extracted from the cell population. In the present specification, the mean value of the sizes of the cell aggregations and the aspect ratios are not limited to those calculated using all the cell aggregations constituting the cell population, and also mean the above-described estimated values. As described above, it should be noted that the mean values of the sizes and aspect ratios of the large number of cell aggregations constituting the cell population are values calculated under the condition of removing the cells and cell aggregations having a size less than the predetermined cut-off value, for example, 30 μm.

### [Standard Deviation of Sizes and Aspect Ratios of a Large Number of Cell Aggregations]

In the present specification, the standard deviation related to the sizes and the aspect ratios may be a population standard deviation or an estimated value thereof. A population standard deviation σ is defined as follows.

$$σ = {( \sum {( μ - {x}_{i} )}^{2} / n )}^{1 / 2}$$

Herein, “u” is the mean value of the sizes or aspect ratios of the large number of cell aggregations. “n” is defined by the number of all cell aggregations constituting the cell population. “xᵢ” means the size or aspect ratio of an i-th cell aggregation. “i” is a natural number of 1 to n. The sigma symbol (E) means summing 1 to n for “i”.

The standard deviation related to the sizes and the aspect ratios may be defined by an estimated value of the population standard deviation instead of the population standard deviation. The estimated value of the population standard deviation is defined by the following equation using some cell aggregations (samples) constituting the cell population.

$$σ = {( \sum {( μ - {x}_{i} )}^{2} / ( n - 1 ) )}^{1 / 2}$$

Herein, “μ” is the mean value of the sizes or aspect ratios of a large number of cell aggregations (samples). “n” is defined by the number of some cell aggregations (samples) constituting the cell population. “n” is the same value as the number of cell aggregations used for calculating the mean value of the sizes or the aspect ratios. “xᵢ” means the size or aspect ratio of an i-th cell aggregation in the samples. “i” is a natural number of 1 to n. The sigma symbol (E) means summing 1 to n for “i”.

In the present specification, the standard deviations of the mean value of the sizes of the cell aggregations and the aspect ratios are not limited to the population standard deviations calculated using all the cell aggregations constituting the cell population, and also mean the above-described estimated values of the population standard deviations, that is, the estimated values calculated on the basis of the samples.

In the present specification, the “large number of cell aggregations” may be, for example, 2 or more, preferably 4 or more, more preferably 10 or more, and still more preferably 100 or more cell aggregations. In this case, the above-described value “n” may similarly be, for example, 2, preferably 4, more preferably 10, and still more preferably 100.

### [Coefficient of Variation]

The coefficient of variation related to the sizes and aspect ratios of the large number of cell aggregations constituting the cell population is defined by a value (o/u) obtained by dividing the standard deviation of each of the sizes and aspect ratios of the large number of cell aggregations by the mean value of each of the sizes and aspect ratios of the large number of cell aggregations. Herein, as described above, the standard deviation may be the population standard deviation or the estimated value of the population standard deviation. Therefore, in the present specification, the coefficient of variation related to the mean value of the sizes of the cell aggregations and the aspect ratios is not limited to the value calculated using all the cell aggregations constituting the cell population, and also means the estimated value calculated using some cell aggregations constituting the cell population.

### [Method 1 for Producing Cell Population]

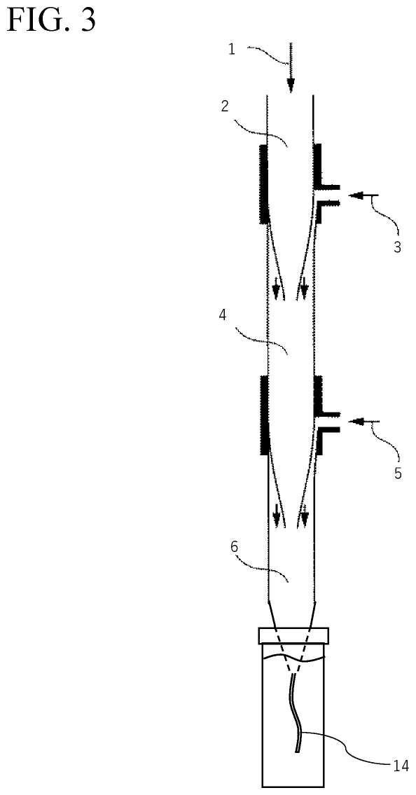

The cell population can be produced, for example, as follows. Herein, a case where the hydrogel encapsulating the cell aggregations has a continuously extending tubular shape as illustrated in FIGS. 1 and 2 will be described. Herein, FIG. 3 is a schematic view illustrating an example of a device for producing the cell population.

First, a cell suspension 1 constituting the core and a hydrogel precursor 3 constituting the shell are prepared. The material of the hydrogel precursor is as described above. The material of the hydrogel precursor is as described above.

The cell suspension may contain a liquid or sol material constituting the above-described core and cells for forming a cell aggregation. The cells prepared before forming the hydrogel may be in a single cell state or in a cell aggregation state. Preferably, the cells prepared before forming the hydrogel are in a single cell state.

The cell suspension 1 is preferably caused to flow as a laminar flow. The flow of the cell suspension 1 is formed in a first introduction pipe 2. The hydrogel precursor 3 is caused to flow over the periphery of the flow of the cell suspension 1. That is, the hydrogel precursor 3 flows coaxially with the cell suspension 1 in the same direction. Preferably, the hydrogel precursor 3 is caused to flow as a laminar flow. As a result, the flow of the hydrogel precursor 3 surrounding the flow of the cell suspension 1 is formed at a second introduction pipe 4.

The hydrogel precursor 3 is gelled to form a tubular hydrogel covering the cell suspension 1. This can be realized by bringing a solution (sheath solution) 5 containing a gelling agent for gelling the hydrogel precursor into contact with the periphery of the flow of the hydrogel precursor 3. In the aspect illustrated in FIG. 3, the solution 5 surrounds the periphery of the hydrogel precursor 3 at a third introduction pipe 6. That is, the solution 5 flows coaxially with the cell suspension 1 and the hydrogel precursor 3 in the same direction.

The cell suspension 1, the hydrogel precursor 3, and the solution 5 containing the gelling agent may start flowing in any order at the start and may stop flowing in any order at the stop. However, from the viewpoint of confining the cells without leakage, it is preferable that the cell suspension 1 starts flowing finally at the time of starting the production, and it is desirable that the cell suspension 1 stops flowing firstly at the time of stopping the production. The flow velocity of each of the cell suspension 1, the hydrogel precursor 3, and the solution 5 containing the gelling agent is not particularly limited as long as the shell can be formed by the hydrogel.

The cell suspension 1, the hydrogel precursor 3, and the solution 5 containing the gelling agent flow out of the third introduction pipe 6 and are immersed in a liquid such as a physiological saline solution or a liquid culture medium, or a suspension. Herein, the hydrogel precursor 3 flows out of the third introduction pipe 6 while being gelled by application of the gelling agent. This forms the tubular hydrogel encapsulating the cells.

In the aspect illustrated in FIG. 3, the flow of the cell suspension 1, the flow of the hydrogel precursor 3, and the flow of the solution 5 containing the gelling agent are formed, and flow out of the third introduction pipe 6 to form the tubular hydrogel. Alternatively, the tubular hydrogel encapsulating the cells is also formed by forming the flow of the cell suspension 1, forming the flow of the hydrogel precursor 3 covering the periphery of the first laminar flow, and discharging the two layers of flow into a container accommodating the solution 5 containing the gelling agent.

Subsequently, the cells encapsulated in the tubular hydrogel are cultured. The cells are immersed in a culture solution and cultured in the state of being encapsulated in the hydrogel. The cells can proliferate to form a cell aggregation (spheroid) in the state of being encapsulated in the hydrogel. The cell aggregation can increase in size with culture. As a result, a large number of cell aggregations are formed in the tubular hydrogel.

The culture of the cells or the cell aggregations may be static culture or dynamic culture such as shaking culture. Cells or cell aggregations in a state of not being encapsulated in the hydrogel are typically likely to be deformed by dynamic culture. In the state where the cells or the cell aggregations are encapsulated in the hydrogel, the cells or the cell aggregations are hardly deformed even in the dynamic culture. Therefore, the variation in the sizes and aspect ratios of the large number of cell aggregations constituting the cell population is kept small.

The cell population according to one aspect is constituted by such a large number of cell aggregations and/or a combination of the large number of cell aggregations and the hydrogel. The sizes and aspect ratios of the large number of cell aggregations in the hydrogel are affected by the composition of the cell suspension (in particular, the concentration of an extracellular matrix), the initial density of the cells (hereinafter, may be referred to as “initial cell density”) contained in the cell suspension forming the core, the size (in particular, the inner diameter) of the shell at the initial stage of hydrogel formation, the number of days of culture of the cells, and the like. Therefore, the composition of the cell suspension (in particular, the concentration of the extracellular matrix), the initial cell density, the inner diameter of the shell, the number of days of culture of the cells, and the like only have to be appropriately set so that the mean value, the standard deviation, the coefficient of variation, and/or the like of the sizes and/or aspect ratios of the large number of cell aggregations have the above-described desired values.

As a result, the cell population including the hydrogel and the large number of cell aggregations in the hydrogel as described above is produced. The specific aspect of the cell population is as described above, and the description thereof may be omitted here.

In one example, a method for producing the cell population according to one aspect may include: encapsulating the cells in the tubular shell formed by the hydrogel; and culturing the cells in the shell so that after removing the cells and cell aggregations having a size less than the predetermined cut-off value, the coefficient of variation obtained by dividing the standard deviation of the sizes of the large number of cell aggregations by the mean value of the sizes of the large number of cell aggregations is 0.50 or less, and/or the coefficient of variation obtained by dividing the standard deviation of the aspect ratios of the shapes of the large number of cell aggregations by the mean value of the aspect ratios of the shapes of the large number of cell aggregations is 0.30 or less. More preferable numerical ranges of these coefficients of variation are as described above.

In the method for producing the cell population, the outer diameter and inner diameter of the shell formed by the hydrogel can be changed by changing the ratio of the flow velocities of the flow of the cell suspension 1, the flow of the hydrogel precursor 3, and the flow of the solution 5 containing the gelling agent.

The composition of the cell suspension (particularly, the concentration of the extracellular matrix) and the density of the cells contained in the cell suspension (initial cell density) only have to be set so that the sizes and aspect ratios of the cell aggregations formed in the hydrogel, and/or the mean value or standard deviation thereof and the coefficient of variation thereof have the desired values.

For example, in a case where the concentration of the extracellular matrix in the cell suspension is very high, the cell aggregations are difficult to form. The higher the initial cell density of the cells contained in the cell suspension and the smaller the inner diameter of the shell, the larger the ratio of the size of each cell aggregation to the inner diameter of the shell tends to be. In this case, it is considered that the sizes of the large number of cell aggregations are restricted by the inner diameter of the shell and as a result tend to be uniform. Therefore, from the viewpoint of reducing the coefficient of variation obtained by dividing the standard deviation of the sizes of the large number of cell aggregations by the mean value of the sizes of the large number of cell aggregations, it is preferable that the extracellular matrix concentration in the cell suspension is low (may be zero), the initial cell density of the cells contained in the cell suspension is high, and/or the inner diameter of the shell is small.

From the viewpoint of reducing the variation in the sizes of the cell aggregations, the initial cell density may be, for example, 5×10⁵ cells/mL or more, preferably 1×10⁶ cells/mL or more, and more preferably 2×10⁶ cells/mL or more. From the viewpoint of reducing the variation in the sizes of the cell aggregations, the initial cell density may be a higher value. Similarly, the initial inner diameter of the shell may be, for example, 400 μm or less, preferably 300 μm or less, and more preferably 250 μm or less. From the viewpoint of reducing the variation in the sizes of the cell aggregations, the initial inner diameter of the shell may be a value smaller than that in the example.

In addition, in a case where the cell suspension contains the extracellular matrix, the cell aggregations are linked depending on the concentration thereof, and the aspect ratios of the cell aggregations tend to be large and nonuniform. In addition, as the cell suspension has a higher viscosity (the thickener has a higher concentration), the distribution of the aspect ratios of the shapes of the large number of cell aggregations tends to be sharper. The lower the initial cell density of the cells contained in the cell suspension and the larger the inner diameter of the shell, the smaller the ratio of the size of each cell aggregation to the inner diameter of the shell tends to be. In this case, it is considered that the large number of cell aggregations are less restricted by the inner diameter of the shell, so that the aspect ratios of the shapes of the large number of cell aggregations tend to be uniform. Therefore, from the viewpoint of reducing the coefficient of variation obtained by dividing the standard deviation of the aspect ratios of the shapes of the large number of cell aggregations by the mean value of the aspect ratios of the shapes of the large number of cell aggregations, it is preferable that the extracellular matrix concentration in the cell suspension is low (may be zero), the viscosity is high, the initial cell density of the cells contained in the cell suspension is low, and the inner diameter of the shell is large.

From the viewpoint of reducing the variation in the aspect ratios of the cell aggregations, the initial cell density may be, for example, 1×10⁸ cells/mL or less, and preferably 5×10⁷ cells/mL or less. From the viewpoint of reducing the variation in the aspect ratios of the cell aggregations, the initial cell density may be a lower value. Similarly, the initial inner diameter of the shell may be, for example, 40 μm or more, and preferably 80 μm or more. From the viewpoint of reducing the variation in the aspect ratios of the cell aggregations, the initial inner diameter of the shell may be a value larger than that in the example.

### [Method 2 for Producing Cell Population]

The cell population according to another aspect may include a large number of cell aggregations collected by removing the hydrogel encapsulating the large number of cell aggregations constituting the cell population described above. Specifically, this cell population can be produced by removing the hydrogel produced by the method 1 for producing the cell population and collecting the cell aggregations in the hydrogel.

Therefore, a method for producing the cell population according to another aspect includes: encapsulating the cells in the tubular shell formed by the hydrogel; culturing the cells in the shell to form the large number of cell aggregations; and collecting the large number of cell aggregations from inside the shell to obtain the cell population in which after removing the cells and cell aggregations having a size less than the predetermined cut-off value, the coefficient of variation obtained by dividing the standard deviation of the sizes of the large number of cell aggregations by the mean value of the sizes of the large number of cell aggregations is 0.50 or less, and/or the coefficient of variation obtained by dividing the standard deviation of the aspect ratios of the shapes of the large number of cell aggregations by the mean value of the aspect ratios of the shapes of the large number of cell aggregations is 0.30 or less. More preferable numerical ranges of these coefficients of variation are as described above.

Herein, the hydrogel can be removed by, for example, a chemical reaction and an enzyme reaction. Specifically, the hydrogel can be removed by, for example, an EDTA/PBS solution or an alginate lyase. The cell population can be obtained by collecting the cell aggregations after removing the hydrogel.

As described above, it has been found that the sizes and aspect ratios of the large number of cell aggregations collected from the hydrogel, the variation in these values, and the like do not significantly change in the large number of collected cell aggregations (cell population). Therefore, in order to obtain a desired cell population, the hydrogel only has to be removed at the timing when the sizes, aspect ratios, and the like of the large number of cell aggregations in the hydrogel reach the above-described desired values during culture of the cells or the cell aggregations in the hydrogel.

In order to collect the cell aggregations from the inside of the hydrogel without changing the sizes and aspect ratios of the cell aggregations much, for example, a solution containing EDTA or an alginate lyase can be used for the removal of the hydrogel. An EDTA/PBS solution can be preferably used. In this case, the concentration of EDTA in the EDTA/PBS solution may be, for example, 50 mM or less, preferably 20 mM or less, more preferably 10 mM or less, and still more preferably 5 mM or less from the viewpoint of collecting the cell aggregations without changing the sizes or the aspect ratios much.

### [Biological Substance]

It should be noted that a biological substance generated by the large number of cell aggregations constituting the cell population described above are also included in the present invention. Herein, the biological substance may be any substance generated by the cells or the cell aggregations. The biological substance may be, for example, a macromolecule such as a nucleic acid, a protein, or a polysaccharide. Such a biological substance can be generated during culture of the above-described cells or cell population.

Such a biological substance may be generated by the cells or the cell aggregations in the state of being encapsulated in the hydrogel. Alternatively, the biological substance may be generated by the large number of cell aggregations collected from the hydrogel.

### Example 1

Example 1 will be described in detail. The following reagents were prepared.
Human amnion-derived mesenchymal stem cells (PromoCell)
Reagent for core: 0.3 w/v % methylcellulose solution (R&D scientific Inc.)
Alginic acid derivative: sodium alginate (“High-G ALG300” manufactured by KIMICA Corporation)
Culture medium: MSC growth medium 2 (PromoCell)

An alginic acid derivative used had an approximate molecular weight of 1,650,000 to 2,050,000, and had an M/G ratio of 0.8 or less.

First, a cell suspension, an alginic acid derivative (hydrogel derivative), and a sheath solution were prepared. The sheath solution is an aqueous solution containing 100 mM calcium chloride and 3 w/v % sucrose.

The alginic acid derivative (hydrogel precursor) is a sodium alginate solution. The sodium alginate solution was generated by adding the above sodium alginate to a physiological saline solution and stirring the obtained solution. The concentration of the sodium alginate relative to the physiological saline solution was 1.44 mass percent concentration.

The cell suspension was obtained by introducing the above human amnion-derived mesenchymal stem cells into a 0.3 w/v % methylcellulose solution (solvent: MSC growth medium 2). The density (initial cell density) of the human amnion-derived mesenchymal stem cells in the cell suspension was 2×10⁶ cells/mL as shown in Table 1 below. The cells introduced into the methylcellulose solution were cells in a single cell form.

Using the above cell suspension, alginic acid derivative (hydrogel precursor), and sheath solution, a cell population was produced on the basis of the “Method 1 for producing cell population” described above (see also FIG. 3). That is, the flow of the cell suspension, the flow of the alginic acid derivative around the flow of the cell suspension, and the flow of the sheath solution around the flow of the alginic acid derivative were formed, and these flows were discharged into a physiological saline solution. The alginic acid derivative was crosslinked by contact with the aqueous calcium chloride solution as the sheath solution to form a tubular alginate gel.

As a result, a tubular hydrogel encapsulating the human amnion-derived mesenchymal stem cells was generated in the physiological saline solution. The outer diameter and inner diameter of the tubular hydrogel (shell) generated are shown in Table 1 below. The outer diameter and inner diameter of the shell shown in Table 1 are values immediately after formation of the shell. The outer diameter and inner diameter of the shell shown in Table 1 can increase with swelling of the hydrogel and an increase in the size of a cell aggregation in the shell.

The tubular hydrogel encapsulating the cells was left still in the physiological saline solution for a desired time. Subsequently, the tubular hydrogel encapsulating the cells was transferred to a liquid culture medium, and the cells were cultured in the tubular hydrogel.

The cells were cultured together with the hydrogel in the liquid culture medium in the state of being encapsulated in the hydrogel. The culture method was static culture. The cells were cultured at a temperature of 37° C. for 4 days. The liquid culture medium used for culture is the above-described MSC growth medium 2.

Through the above steps, a large number of cell aggregations were generated in the hydrogel. The cell population in Example 1 is constituted by the hydrogel and the large number of cell aggregations encapsulated in this hydrogel.

### Example 2

Next, a cell population according to Example 2 will be described. The cell population according to Example 2 was produced in the same manner as in Example 1 except for the flow velocity of the sheath solution. The flow velocity of the sheath solution was faster than that in the case of Example 1. By changing the flow velocity of the sheath solution, the outer diameter and inner diameter of the shell immediately after formation of the shell in Example 2 are different from those in Example 1. The outer diameter and inner diameter of the tubular hydrogel (shell) generated in Example 2 are shown in Table 1 below.

The cells were cultured together with the hydrogel in the liquid culture medium in the state of being encapsulated in the hydrogel in Example 2 as well. The conditions for cell culture are the same as those in Example 1. Through the above steps, a large number of cell aggregations were generated in the hydrogel. The cell population in Example 2 is constituted by the hydrogel and the large number of cell aggregations encapsulated in this hydrogel.

### Example 3

Next, a cell population according to Example 3 will be described. The cell population according to Example 3 was produced in the same manner as in Example 1 except for the flow velocity of the sheath solution. The flow velocity of the sheath solution was faster than those in the cases of Examples 1 and 2. By changing the flow velocity of the sheath solution, the outer diameter and inner diameter of the shell immediately after formation of the shell in Example 3 are different from those in Examples 1 and 2. The outer diameter and inner diameter of the tubular hydrogel (shell) generated in Example 3 are shown in Table 1 below.

The cells were cultured together with the hydrogel in the liquid culture medium in the state of being encapsulated in the hydrogel in Example 3 as well. The conditions for cell culture are the same as those in Example 1. Through the above steps, a large number of cell aggregations were generated in the hydrogel. The cell population in Example 3 is constituted by the hydrogel and the large number of cell aggregations encapsulated in this hydrogel.

### Example 4

Next, a cell population according to Example 4 will be described. The cell population according to Example 4 was produced in the same manner as in Example 1 except for the density (initial cell density) of the cells in the cell suspension before encapsulation in the shell. In Example 4, the density (initial cell density) of the human amnion-derived mesenchymal stem cells in the cell suspension was 1×10⁷ cells/mL as shown in Table 1 below. The outer diameter and inner diameter of the tubular hydrogel (shell) generated in Example 4 are shown in Table 1 below.

The cells were cultured together with the hydrogel in the liquid culture medium in the state of being encapsulated in the hydrogel in Example 4 as well. The conditions for cell culture are the same as those in Example 1. Through the above steps, a large number of cell aggregations were generated in the hydrogel. The cell population in Example 4 is constituted by the hydrogel and the large number of cell aggregations encapsulated in this hydrogel.

### Example 5

Next, a cell population according to Example 5 will be described. The cell population according to Example 5 was produced in the same manner as in Example 2 except for the density (initial cell density) of the cells in the cell suspension before encapsulation in the shell. In Example 5, the density (initial cell density) of the human amnion-derived mesenchymal stem cells in the cell suspension was 1×10⁷ cells/mL as shown in Table 1 below. The outer diameter and inner diameter of the tubular hydrogel (shell) generated in Example 5 are shown in Table 1 below.

The cells were cultured together with the hydrogel in the liquid culture medium in the state of being encapsulated in the hydrogel in Example 5 as well. The conditions for cell culture are the same as those in Example 1. Through the above steps, a large number of cell aggregations were generated in the hydrogel. The cell population in Example 5 is constituted by the hydrogel and the large number of cell aggregations encapsulated in this hydrogel.

### Example 6

Next, a cell population according to Example 6 will be described. The cell population according to Example 6 was produced in the same manner as in Example 3 except for the density (initial cell density) of the cells in the cell suspension before encapsulation in the shell. In Example 6, the density (initial cell density) of the human amnion-derived mesenchymal stem cells in the cell suspension was 1×10⁷ cells/mL as shown in Table 1 below. The outer diameter and inner diameter of the tubular hydrogel (shell) generated in Example 6 are shown in Table 1 below.

The cells were cultured together with the hydrogel in the liquid culture medium in the state of being encapsulated in the hydrogel in Example 6 as well. The conditions for cell culture are the same as those in Example 1. Through the above steps, a large number of cell aggregations were generated in the hydrogel. The cell population in Example 6 is constituted by the hydrogel and the large number of cell aggregations encapsulated in this hydrogel.

### Example 7

Next, a cell population according to Example 7 will be described. The cell population according to Example 7 was produced in the same manner as in Example 1 except for the density (initial cell density) of the cells in the cell suspension before encapsulation in the shell. In Example 7, the density (initial cell density) of the human amnion-derived mesenchymal stem cells in the cell suspension was 5×10⁷ cells/mL as shown in Table 1 below. The outer diameter and inner diameter of the tubular hydrogel (shell) generated in Example 7 are shown in Table 1 below.

The cells were cultured together with the hydrogel in the liquid culture medium in the state of being encapsulated in the hydrogel in Example 7 as well. The conditions for cell culture are the same as those in Example 1. Through the above steps, a large number of cell aggregations were generated in the hydrogel. The cell population in Example 7 is constituted by the hydrogel and the large number of cell aggregations encapsulated in this hydrogel.

### Example 8

Next, a cell population according to Example 8 will be described. The cell population according to Example 8 was produced in the same manner as in Example 2 except for the density (initial cell density) of the cells in the cell suspension before encapsulation in the shell. In Example 8, the density (initial cell density) of the human amnion-derived mesenchymal stem cells in the cell suspension was 5×10⁷ cells/mL as shown in Table 1 below. The outer diameter and inner diameter of the tubular hydrogel (shell) generated in Example 8 are shown in Table 1 below.

The cells were cultured together with the hydrogel in the liquid culture medium in the state of being encapsulated in the hydrogel in Example 8 as well. The conditions for cell culture are the same as those in Example 1. Through the above steps, a large number of cell aggregations were generated in the hydrogel. The cell population in Example 8 is constituted by the hydrogel and the large number of cell aggregations encapsulated in this hydrogel.

### Example 9

Next, a cell population according to Example 9 will be described. The cell population according to Example 9 was produced in the same manner as in Example 3 except for the density (initial cell density) of the cells in the cell suspension before encapsulation in the shell. In Example 9, the density (initial cell density) of the human amnion-derived mesenchymal stem cells in the cell suspension was 5×10⁷ cells/mL as shown in Table 1 below. The outer diameter and inner diameter of the tubular hydrogel (shell) generated in Example 9 are shown in Table 1 below.

The cells were cultured together with the hydrogel in the liquid culture medium in the state of being encapsulated in the hydrogel in Example 9 as well. The conditions for cell culture are the same as those in Example 1. Through the above steps, a large number of cell aggregations were generated in the hydrogel. The cell population in Example 9 is constituted by the hydrogel and the large number of cell aggregations encapsulated in this hydrogel.

### Reference Example 1

As Reference Example 1, dispersed cells and a culture medium were added to a cell culture plate (6 well plate) without using a hydrogel, and static culture was performed. 2×10⁵ cells were suspended in a 2 mL culture medium. The cells are the above-described human amnion-derived mesenchymal stem cells. The culture medium is the above-described MSC growth medium 2. The cells were cultured at a temperature of 37° C. for 4 days.

Through the above steps, a large number of cell aggregations were generated in the cell culture plate. A cell population in Reference Example 1 is constituted by the large number of cell aggregations cultured in this cell culture plate.

The mean value of the sizes of the large number of cell aggregations constituting the cell population in Examples 1 to 9 described above and Reference Example 1, the standard deviation thereof, and the coefficient of variation were calculated. The mean value of the sizes of the cell aggregations and the standard deviation thereof were calculated in the state of being encapsulated in the hydrogel in Examples 1 to 9. The mean value of the sizes of the cell aggregations and the standard deviation thereof were calculated by the methods described above. Herein, it should be noted that these numerical values are calculated from values measured for a large number of cell aggregations (samples) randomly extracted from the large number of cell aggregations constituting the cell population, in consideration of the above-described cut-off value. These results are shown in Table 1 below. Herein, in each Example, the distribution of the sizes of the large number of cell aggregations had a shape with a peak near the mean value of the sizes.

TABLE 1

| | Outer | Inner | Initial | Mean Size | Standard Deviation | |
|---|---|---|---|---|---|---|
| | Diameter | Diameter | Cell | of Cell | of Sizes of Cell | Coefficient |
| | of Shell | of Shell | Density | Aggregations | Aggregations | of Variation |
| | (μm) | (μm) | (cells/mL) | μ (μm) | σ (μm) | σ/μ |
| Example 1 | 517 | 232 | 2 × 10⁶ | 69.4 | 18.5 | 0.27 |
| Example 2 | 264 | 119 | 2 × 10⁶ | 67.5 | 15.5 | 0.23 |
| Example 3 | 184 | 84 | 2 × 10⁶ | 49.3 | 11.4 | 0.23 |
| Example 4 | 489 | 229 | 1 × 10⁷ | 103.1 | 23.7 | 0.23 |
| Example 5 | 266 | 112 | 1 × 10⁷ | 67.9 | 15.5 | 0.23 |
| Example 6 | 181 | 82 | 1 × 10⁷ | 58.3 | 12.7 | 0.22 |
| Example 7 | 506 | 229 | 5 × 10⁷ | 113.0 | 35.6 | 0.32 |
| Example 8 | 266 | 118 | 5 × 10⁷ | 89.1 | 17.8 | 0.20 |
| Example 9 | 180 | 80 | 5 × 10⁷ | 69.2 | 15.8 | 0.23 |
| Reference | — | — | — | 111.6 | 70.8 | 0.63 |
| Example 1 | | | | | | |

The coefficient of variation, which is a value obtained by dividing the mean value by the standard deviation, is used as an index indicating the spread of the distribution. That is, the sharper the distribution, the smaller the coefficient of variation.

Table 1 shows that the coefficient of variation obtained by dividing the standard deviation of the sizes of the large number of cell aggregations constituting the cell population by the mean value of the sizes of the large number of cell aggregations in all Examples 1 to 9 is much smaller than that in Reference Example 1. Therefore, in all Examples 1 to 9, the distribution of the sizes of the large number of cell aggregations constituting the cell population is sharp. In other words, in Examples 1 to 9, the sizes of the large number of cell aggregations constituting the cell population are uniformized.

As described above, from the viewpoint of reducing the variation in the sizes of the cell aggregations, the initial cell density is preferably as high as possible. In Examples 1 to 3, the initial cell density is 2×10⁶ cells/mL. In the cases of Examples 1 to 3, the coefficient of variation obtained by dividing the standard deviation of the sizes of the large number of cell aggregations constituting the cell population by the mean value of the sizes of the large number of cell aggregations is 0.23 or 0.27. Therefore, assuming that the allowable maximum value of the coefficient of variation is 0.50, it is considered that the initial cell density may be a value lower than 2×10⁶ cells/mL, for example, may be about 5×10⁵ cells/mL.

From the viewpoint of reducing the variation in the sizes of the cell aggregations, the initial inner diameter of the shell is preferably as small as possible. In Examples 1, 4, and 7, the initial inner diameter of the shell is 232 μm, 229 μm, and 229 μm, respectively. In the cases of Examples 1, 4, and 7, the coefficient of variation obtained by dividing the standard deviation of the sizes of the large number of cell aggregations constituting the cell population by the mean value of the sizes of the large number of cell aggregations is 0.27, 0.23, and 0.32, respectively. Assuming that the allowable maximum value of the coefficient of variation is 0.50, it is considered that the initial inner diameter of the shell may be a value larger than 232 μm, for example, may be about 1,000 μm or 500 μm.

As described above, it is considered that the higher the initial cell density of the cells contained in the cell suspension and the smaller the inner diameter of the shell, the smaller the coefficient of variation obtained by dividing the standard deviation of the sizes of the large number of cell aggregations by the mean value of the sizes of the large number of cell aggregations tends to be. In consideration of such a viewpoint and the results shown in Table 1, it is found that, for example, when the initial cell density is 2×10⁶ cells/mL or more and the inner diameter of the shell is 250 μm or less, the variation in the sizes of the cell aggregations can be sufficiently reduced. However, it should be noted that the present invention is not limited to these numerical ranges.

In addition, the mean value of the aspect ratios of the large number of cell aggregations constituting the cell population in Examples 1 to 9 described above and Reference Example 1, the standard deviation thereof, and the coefficient of variation were calculated. The mean value of the aspect ratios of the cell aggregations and the standard deviation thereof were calculated in the state of being encapsulated in the hydrogel in Examples 1 to 9. The mean value of the aspect ratios of the cell aggregations and the standard deviation thereof were calculated by the methods described above. Herein, it should be noted that these numerical values are calculated from values measured for a large number of cell aggregations (samples) randomly extracted from the large number of cell aggregations constituting the cell population, in consideration of the above-described cut-off value. The calculated mean value of the aspect ratios of the cell aggregations and the standard deviation thereof are shown in Table 2 below. Herein, in each Example, the distribution of the aspect ratios of the large number of cell aggregations had a shape with a peak near the mean value of the aspect ratios.

TABLE 2

| | Outer | Inner | Initial | Mean Value of | Standard Deviation | |
|---|---|---|---|---|---|---|
| | Diameter | Diameter | Cell | Aspect Ratios of | of Aspect Ratios of | Coefficient |
| | of Shell | of Shell | Density | Cell Aggregations | Cell Aggregations | of Variation |
| | (μm) | (μm) | (cells/mL) | μ | σ | σ/μ |
| Example 1 | 517 | 232 | 2 × 10⁶ | 1.19 | 0.16 | 0.13 |
| Example 2 | 264 | 119 | 2 × 10⁶ | 1.26 | 0.21 | 0.17 |
| Example 3 | 184 | 84 | 2 × 10⁶ | 1.16 | 0.14 | 0.12 |
| Example 4 | 489 | 229 | 1 × 10⁷ | 1.18 | 0.15 | 0.13 |
| Example 5 | 266 | 112 | 1 × 10⁷ | 1.19 | 0.16 | 0.13 |
| Example 6 | 181 | 82 | 1 × 10⁷ | 1.32 | 0.29 | 0.22 |
| Example 7 | 506 | 229 | 5 × 10⁷ | 1.14 | 0.10 | 0.09 |
| Example 8 | 266 | 118 | 5 × 10⁷ | 1.15 | 0.11 | 0.10 |
| Example 9 | 180 | 80 | 5 × 10⁷ | 1.29 | 0.25 | 0.20 |
| Reference | — | — | — | 1.37 | 0.44 | 0.32 |
| Example 1 | | | | | | |

Table 2 shows that the coefficient of variation obtained by dividing the standard deviation of the aspect ratios of the shapes of the large number of cell aggregations constituting the cell population by the mean value of the aspect ratios of the shapes of the large number of cell aggregations in all Examples 1 to 9 is significantly smaller than that in Reference Example 1. Therefore, in all Examples 1 to 9, the distribution of the aspect ratios of the shapes of the large number of cell aggregations constituting the cell population is sharp. In other words, in Examples 1 to 9, the shapes of the large number of cell aggregations constituting the cell population are uniformized.

As described above, from the viewpoint of reducing the variation in the aspect ratios of the cell aggregations, the initial cell density is preferably as low as possible. In Examples 7 to 9, the initial cell density is 5×10⁷ cells/mL. In the cases of Examples 7 to 9, the coefficient of variation obtained by dividing the standard deviation of the aspect ratios of the large number of cell aggregations constituting the cell population by the mean value of the aspect ratios of the large number of cell aggregations is 0.09, 0.10, and 0.20, respectively. Therefore, assuming that the allowable maximum value of the coefficient of variation is 0.30, it is considered that the initial cell density may be a value higher than 5×10⁷ cells/mL, for example, may be about 1×10⁸ cells/mL.

From the viewpoint of reducing the variation in the aspect ratios of the cell aggregations, the initial inner diameter of the aspect ratio is preferably as large as possible. In Examples 3, 6, and 9, the initial inner diameter of the shell is 84 μm, 82 μm, and 80 μm, respectively. In the cases of Examples 3, 6, and 9, the coefficient of variation obtained by dividing the standard deviation of the aspect ratios of the large number of cell aggregations constituting the cell population by the mean value of the aspect ratios of the large number of cell aggregations is 0.12, 0.22, and 0.20, respectively. Assuming that the allowable maximum value of the coefficient of variation is 0.30, it is considered that the initial inner diameter of the shell may be a value smaller than 80 μm, for example, may be about 40 μm.

As described above, it is considered that the lower the initial cell density of the cells contained in the cell suspension and the larger the inner diameter of the shell, the smaller the coefficient of variation obtained by dividing the standard deviation of the aspect ratios of the shapes of the large number of cell aggregations by the mean value of the aspect ratios of the shapes of the large number of cell aggregations tends to be. In consideration of such a viewpoint and the results shown in Table 2, it is found that, for example, when the initial cell density is 5×10⁷ cells/mL or less and the inner diameter of the shell is 60 μm or more, the variation in the aspect ratios of the shapes of the cell aggregations can be sufficiently reduced. However, it should be noted that the present invention is not limited to these numerical ranges.

### Example 10

Example 10 will be described in detail. The following reagents were prepared.
Human bone marrow-derived mesenchymal stem cells (PromoCell)
Reagent for core: 0.3 w/v % methylcellulose solution
Alginic acid derivative: sodium alginate (“High-G ALG300” manufactured by KIMICA Corporation)
Culture medium: MSC growth medium 2 (PromoCell)

First, a cell suspension, an alginic acid derivative (hydrogel derivative), and a sheath solution were prepared. The sheath solution is an aqueous solution containing 100 mM calcium chloride and 3 w/v % sucrose.

The alginic acid derivative (hydrogel precursor) is a sodium alginate solution. The sodium alginate solution was generated by adding the above sodium alginate to a physiological saline solution and stirring the obtained solution. The concentration of the sodium alginate relative to the physiological saline solution was 1.44 mass percent concentration.

The cell suspension was obtained by introducing the above human amnion-derived mesenchymal stem cells into a 0.3 w/v % methylcellulose solution (solvent: MSC growth medium 2 described above). The density (initial cell density) of the human amnion-derived mesenchymal stem cells in the cell suspension was 1×10⁷ cells/mL as shown in Table 3 below. The cells introduced into the methylcellulose solution were cells in a single cell form.

Using the above cell suspension, alginic acid derivative (hydrogel precursor), and sheath solution, a cell population was produced on the basis of the “Method 1 for producing cell population” described above (see also FIG. 3). That is, the flow of the cell suspension, the flow of the alginic acid derivative around the flow of the cell suspension, and the flow of the sheath solution around the flow of the alginic acid derivative were formed, and these flows were discharged into a physiological saline solution. The alginic acid derivative was crosslinked by contact with the aqueous calcium chloride solution as the sheath solution to form a tubular alginate gel.

As a result, a tubular hydrogel encapsulating the human amnion-derived mesenchymal stem cells was generated in the physiological saline solution. The outer diameter and inner diameter of the tubular hydrogel (shell) generated are shown in Table 3 below. The outer diameter and inner diameter of the shell shown in Table 3 are values immediately after formation of the shell. The outer diameter and inner diameter of the shell shown in Table 3 can increase with swelling of the hydrogel and an increase in the size of a cell aggregation in the shell.

The tubular hydrogel encapsulating the cells was left still in the physiological saline solution for a desired time. Subsequently, the tubular hydrogel encapsulating the cells was transferred to a liquid culture medium, and the cells were cultured in the tubular hydrogel.

The cells were cultured together with the hydrogel in the liquid culture medium in the state of being encapsulated in the hydrogel. The cells were cultured at a temperature of 37° C. for 2 days. The liquid culture medium used for culture is the above-described MSC growth medium 2.

Through the above steps, a large number of cell aggregations were generated in the hydrogel. In Example 10, four tubular hydrogels were produced under the same conditions. As a result, four cell populations were produced. These four cell populations are distinguished in Table 3 as “Example 10 fiber 1”, “Example 10_fiber 2”, “Example 10 fiber 3”, and “Example 10 fiber 4”. The outer diameters and inner diameters of these hydrogels were 260±15 μm and 108±14 μm, respectively.

### Example 11

Example 11 will be described in detail. A cell population of Example 11 is constituted by a large number of cell aggregations collected from the inside of the hydrogel by removing the hydrogel in “Example 10_fiber 1” described above.

Specifically, first, the supernatant of the culture medium in which the hydrogel in “Example 10 fiber 1” was immersed was removed, and the hydrogel was washed with 2 mL phosphate-buffered saline (PBS). Subsequently, the hydrogel with the large number of cell aggregations was immersed in an EDTA-PBS solution having a hydrogen ion exponent (pH) of 7, and treated at room temperature for 5 minutes. The EDTA concentration of the EDTA-PBS solution was 10 mM. After this treatment, the cell aggregations were centrifuged at 4° C. for 5 minutes, and the precipitated cell aggregations were suspended in the culture medium and collected. The cell population of Example 11 is constituted by the large number of cell aggregations collected in this way.

### Example 12

Example 12 will be described in detail. A cell population of Example 12 is constituted by a large number of cell aggregations collected from the inside of the hydrogel by removing the hydrogel in “Example 10_fiber 2” described above.

The method of collecting the cell aggregations in the hydrogel is the same as that in Example 11 except for the concentration of the EDTA-PBS solution. In Example 12, the EDTA concentration of the EDTA-PBS solution was 2 mM. The cell population of Example 12 is constituted by the large number of cell aggregations collected in this way.

### Example 13

Example 13 will be described in detail. A cell population of Example 13 is constituted by a large number of cell aggregations collected from the inside of the hydrogel by removing the hydrogel in “Example 10 fiber 3” described above.

The method of collecting the cell aggregations in the hydrogel is the same as that in Example 11 except for the concentration of the EDTA-PBS solution. In Example 13, the EDTA concentration of the EDTA-PBS solution was 1 mM. The cell population of Example 13 is constituted by the large number of cell aggregations collected in this way.

### Example 14

Example 14 will be described in detail. A cell population of Example 11 is constituted by a large number of cell aggregations collected from the inside of the hydrogel by removing the hydrogel in “Example 10_fiber 4” described above.

Specifically, first, an alginate lyase (AL) was added to the culture medium in which the hydrogel in “Example 10 fiber 4” was immersed so that the concentration of the alginate lyase was 0.08 w/v %, without removing the culture supernatant. Then, incubation was performed at 37° C. for 10 minutes. After this treatment, the cell aggregations were centrifuged at 4° C. for 5 minutes, and the precipitated cell aggregations were suspended in the culture medium and collected. The cell population of Example 14 is constituted by the large number of cell aggregations collected in this way.

### Example 15

Next, a cell population according to Example 15 will be described. The cell population according to Example 15 was produced in the same manner as in Example 10 except for the density (initial cell density) of the cells in the cell suspension before encapsulation in the shell. In Example 15, the density (initial cell density) of the human bone marrow-derived mesenchymal stem cells in the cell suspension was 5×10⁷ cells/mL as shown in Table 3 below.

In Example 15, three tubular hydrogels were produced under the same conditions. As a result, three cell populations were produced. These three cell populations are distinguished in Table 3 as “Example 15 fiber 1”, “Example 15_fiber 2”, and “Example 15 fiber 3”. The outer diameters and inner diameters of these hydrogels were 273±16 μm and 122±12 μm, respectively.

### Example 16

A cell population of Example 16 is constituted by a large number of cell aggregations collected from the inside of the hydrogel by removing the hydrogel in “Example 15 fiber 1” described above. The method of collecting the cell aggregations from the hydrogel is the same as that in Example 11.

### Example 17

A cell population of Example 17 is constituted by a large number of cell aggregations collected from the inside of the hydrogel by removing the hydrogel in “Example 15 fiber 2” described above. The method of collecting the cell aggregations from the hydrogel is the same as that in Example 12.

### Example 18

A cell population of Example 18 is constituted by a large number of cell aggregations collected from the inside of the hydrogel by removing the hydrogel in “Example 15 fiber 3” described above. The method of collecting the cell aggregations from the hydrogel is the same as that in Example 13.

### Reference Example 2

As Reference Example 2, dispersed cells and a culture medium were added to a cell culture plate (6 well plate) without using a hydrogel, and static culture was performed. 2×10⁵ cells were suspended in a 2 mL culture medium. The cells are the above-described human bone marrow-derived mesenchymal stem cells. The culture medium is the above-described MSC growth medium 2. The cells were cultured at a temperature of 37° C. for 2 days.

Through the above steps, a large number of cell aggregations were generated in the cell culture plate. A cell population in Reference Example 2 is constituted by the large number of cell aggregations cultured in this cell culture plate.

The mean value of the sizes of the large number of cell aggregations constituting the cell population in Examples 10 to 18 described above and Reference Example 2, the standard deviation thereof, and the coefficient of variation were calculated as described above. In Example 10 and Example 15, the mean value of the sizes of the cell aggregations and the standard deviation thereof were calculated in the state of being encapsulated in the hydrogel. The calculated mean value of the sizes of the cell aggregations and the standard deviation thereof are shown in Table 3 below. Herein, in each Example, the distribution of the sizes of the large number of cell aggregations had a shape with a peak near the mean value of the sizes.

TABLE 3

| | | | | Standard | |
|---|---|---|---|---|---|
| | | | Mean | Deviation | Coef- |
| | Initial | | Size of | of Sizes | ficient |
| | Cell | | Cell | of Cell | of |
| | Density | Reagents for | Aggre- | Aggre- | Vari- |
| | (cells/ | Removing | gations | gations | ation |
| | mL) | Hydrogel | μ (μm) | σ (μm) | σ/μ |
| Example | 1 × 10⁷ | — | 95.16 | 20.71 | 0.22 |
| 10_fiber 1 | | | | | |
| Example | 1 × 10⁷ | — | 93.04 | 24.97 | 0.27 |
| 10_fiber 2 | | | | | |
| Example | 1 × 10⁷ | — | 89.74 | 26.74 | 0.30 |
| 10_fiber 3 | | | | | |
| Example | 1 × 10⁷ | — | 87.47 | 16.21 | 0.19 |
| 10_fiber 4 | | | | | |
| Example 11 | — | 10 mM EDTA | 96.87 | 23.60 | 0.24 |
| Example 12 | — | 2 mM EDTA | 96.67 | 21.29 | 0.22 |
| Example 13 | — | 1 mM EDTA | 86.29 | 20.32 | 0.24 |
| Example 14 | — | 1% AL | 82.96 | 20.43 | 0.25 |
| Example | 5 × 10⁷ | — | 133.61 | 19.87 | 0.15 |
| 15_fiber 5 | | | | | |
| Example | 5 × 10⁷ | — | 144.03 | 26.53 | 0.18 |
| 15_fiber 6 | | | | | |
| Example | 5 × 10⁷ | — | 127.72 | 17.83 | 0.14 |
| 15_fiber 7 | | | | | |
| Example 16 | — | 10 mM EDTA | 140.66 | 22.72 | 0.16 |
| Example 17 | — | 2 mM EDTA | 148.46 | 22.55 | 0.15 |
| Example 18 | — | 1 mM EDTA | 119.03 | 21.68 | 0.18 |
| Reference | — | — | 123.14 | 53.33 | 0.43 |
| Example 2 | | | | | |

Table 3 shows that the coefficient of variation obtained by dividing the standard deviation of the sizes of the large number of cell aggregations constituting the cell population by the mean value of the sizes of the large number of cell aggregations in all Examples 10 to 18 is significantly smaller than that in Reference Example 2. Therefore, in all Examples 10 to 18, the distribution of the sizes of the large number of cell aggregations constituting the cell population is sharp. In other words, in Examples 10 to 18, the sizes of the large number of cell aggregations constituting the cell population are uniformized.

Comparison between Example 10 and Examples 11 to 15 shows that the mean value and standard deviation of the sizes of the large number of cell aggregations collected from the hydrogel, and the coefficient of variation thereof do not significantly change from those values in the state of being encapsulated in the hydrogel. Therefore, with regard to the cell population collected from the hydrogel, the variation in the sizes of the large number of cell aggregations can also be reduced.

Comparison between Example 10 and Example 15 shows that the coefficient of variation obtained by dividing the standard deviation of the sizes of the large number of cell aggregations by the mean value of the sizes of the large number of cell aggregations tends to be smaller as the initial cell density is higher.

In addition, the mean value of the aspect ratios of the large number of cell aggregations constituting the cell population in Examples 10 to 14 described above, the standard deviation thereof, and the coefficient of variation were calculated as described above. The mean value of the aspect ratios of the cell aggregations and the standard deviation thereof were calculated in the state of being encapsulated in the hydrogel. The calculated mean value of the aspect ratios of the cell aggregations and the standard deviation thereof are shown in Table 4 below. Herein, in each Example, the distribution of the aspect ratios of the large number of cell aggregations had a shape with a peak near the mean value of the aspect ratios.

TABLE 4

| | | | Mean Value of | Standard Deviation of | |
|---|---|---|---|---|---|
| | Initial Cell | | Aspect Ratios of | Aspect Ratios of Cell | Coefficient of |
| | Density | Reagent for | Cell Aggregations | Aggregations | Variation |
| | (cells/mL) | Collect | μ | σ | σ/μ |
| Example 10_fiber 1 | 1 × 10⁷ | — | 1.22 | 0.26 | 0.21 |
| Example 10_fiber 2 | 1 × 10⁷ | — | 1.19 | 0.31 | 0.26 |
| Example 10_fiber 3 | 1 × 10⁷ | — | 1.19 | 0.22 | 0.18 |
| Example 10_fiber 4 | 1 × 10⁷ | — | 1.19 | 0.31 | 0.26 |
| Example 11 | — | 10 mM EDTA | 1.44 | 0.42 | 0.29 |
| Example 12 | — | 2 mM EDTA | 1.33 | 0.29 | 0.22 |
| Example 13 | — | 1 mM EDTA | 1.30 | 0.28 | 0.22 |
| Example 14 | — | 1% AL | 1.26 | 0.21 | 0.17 |

Table 4 shows that the coefficient of variation obtained by dividing the standard deviation of the aspect ratios of the shapes of the large number of cell aggregations constituting the cell population by the mean value of the aspect ratios of the shapes of the large number of cell aggregations in Examples 10 to 14 has a relatively small value.

Comparison between Example 10 and Examples 11 to 14 shows that the mean value and standard deviation of the aspect ratios of the large number of cell aggregations collected from the hydrogel, and the coefficient of variation thereof do not significantly change from those values in the state of being encapsulated in the hydrogel. Therefore, with regard to the cell population collected from the hydrogel, the variation in the sizes of the large number of cell aggregations can also be reduced.

### Example 19

Example 19 will be described in detail. The following reagents were prepared.
Human adipose-derived mesenchymal stem cells (PromoCell)
Human iPS cell-derived cardiomyocytes (Myoridge Co., Ltd.)
Reagent for core: 0.3 w/v % methylcellulose solution
Alginic acid derivative: sodium alginate (“High-G ALG300” manufactured by KIMICA Corporation)
Culture medium 1: MSC growth medium 2 (PromoCell)
Culture medium 2: Medium for cardiomyocytes (Myoridge Co., Ltd.)

First, a cell suspension, an alginic acid derivative (hydrogel derivative), and a sheath solution were prepared. The sheath solution is an aqueous solution containing 100 mM calcium chloride and 3 w/v % sucrose.

The alginic acid derivative (hydrogel precursor) is a sodium alginate solution. The sodium alginate solution was generated by adding the above sodium alginate to a physiological saline solution and stirring the obtained solution. The concentration of the sodium alginate relative to the physiological saline solution was 0.99 mass percent concentration.

The cell suspension is a mixture of one obtained by introducing the above human adipose-derived mesenchymal stem cells into a 0.3 w/v % methylcellulose solution (solvent: MSC growth medium 2 described above) and one obtained by introducing the above human iPS cell-derived cardiomyocytes into a 0.3 w/v % methylcellulose solution (solvent: medium for cardiomyocytes). The ratio of the number of human adipose-derived mesenchymal stem cells to the number of human iPS cell-derived cardiomyocytes was 1:1. In Example 19, the density (initial cell density) of both the cells in the cell suspension was 1×10⁷ cells/mL.

Using the above cell suspension, alginic acid derivative (hydrogel precursor), and sheath solution, a cell population was produced on the basis of the “Method 1 for producing cell population” described above (see also FIG. 3). That is, the flow of the cell suspension, the flow of the alginic acid derivative around the flow of the cell suspension, and the flow of the sheath solution around the flow of the alginic acid derivative were formed, and these flows were discharged into a physiological saline solution. The alginic acid derivative was crosslinked by contact with the aqueous calcium chloride solution as the sheath solution to form a tubular alginate gel.

As a result, a tubular hydrogel encapsulating the two types of cells was generated in the physiological saline solution. The outer diameter and inner diameter of the tubular hydrogel (shell) generated are shown in Table 5 below. The outer diameter and inner diameter of the shell shown in Table 5 are values immediately after formation of the shell.

The tubular hydrogel encapsulating the cells was left still in the physiological saline solution for a desired time. Subsequently, the tubular hydrogel encapsulating the cells was transferred to a liquid culture medium, and the cells were cultured in the tubular hydrogel.

The cells were cultured together with the hydrogel in the liquid culture medium in the state of being encapsulated in the hydrogel. The cells were cultured at a temperature of 37° C. for 2 days. The liquid culture medium used for culture is a mixture of the above-described MSC growth medium 2 and the medium for cardiomyocytes to which a ROCK inhibitor is added. In Example 19, the culture method was static culture.

Through the above steps, a large number of cell aggregations were generated in the hydrogel. The cell population in Example 19 is constituted by the hydrogel and the large number of cell aggregations encapsulated in this hydrogel. Herein, it has been able to confirm that each of the cell aggregations in the cell population of Example 19 is the human adipose-derived mesenchymal stem cells and the human iPS cell-derived cardiomyocytes mixed with each other and aggregated together.

Specifically, the human iPS cell-derived cardiomyocytes expressed GFP by introduction of GCaMP, so that only the human iPS cell-derived cardiomyocytes were caused to emit fluorescence. When such cell aggregations were observed with a fluorescence microscope, the cell aggregations were emitting fluorescence in a mottled pattern. Therefore, it is considered that the cell aggregations contained the human iPS cell-derived cardiomyocytes in a mottled manner and contained the human adipose-derived mesenchymal stem cells in another portion.

### Example 20

Next, a cell population according to Example 20 will be described. The cell population according to Example 20 was produced in the same manner as in Example 19 except that the cell suspension forming the core was obtained by introducing the above human iPS cell-derived cardiomyocytes into the 0.3 w/v % methylcellulose solution (solvent: medium for cardiomyocytes). That is, cell aggregations according to Example 20 contain the human iPS cell-derived cardiomyocytes and do not contain the human adipose-derived mesenchymal stem cells.

The outer diameter and inner diameter of the tubular hydrogel (shell) generated in Example 20 are shown in Table 5 below. The cells were cultured together with the hydrogel in the liquid culture medium in the state of being encapsulated in the hydrogel in Example 20 as well. The conditions for cell culture are the same as those in Example 19. Through the above steps, a large number of cell aggregations were generated in the hydrogel.

### Example 21

Next, a cell population according to Example 21 will be described. The cell population according to Example 21 was produced in the same manner as in Example 19 except that the cell suspension forming the core was obtained by introducing the above human adipose-derived mesenchymal stem cells into the 0.3 w/v % methylcellulose solution (solvent: MSC growth medium 2 described above). That is, cell aggregations according to Example 21 contain the human adipose-derived mesenchymal stem cells and do not contain the human iPS cell-derived cardiomyocytes.

The outer diameter and inner diameter of the tubular hydrogel (shell) generated in Example 21 are shown in Table 5 below. The cells were cultured together with the hydrogel in the liquid culture medium in the state of being encapsulated in the hydrogel in Example 21 as well. The conditions for cell culture are the same as those in Example 19. Through the above steps, a large number of cell aggregations were generated in the hydrogel.

### Example 22

Next, a cell population according to Example 22 will be described. The cell population according to Example 22 was produced in the same manner as in Example 19 except for the initial cell density of the human iPS cell-derived cardiomyocytes and the human iPS cell-derived cardiomyocytes. In Example 22, the ratio of the number of human adipose-derived mesenchymal stem cells to the number of human iPS cell-derived cardiomyocytes was 1:1. In Example 22, the density (initial cell density) of both the cells in the cell suspension was 5×10⁷ cells/mL.

The outer diameter and inner diameter of the tubular hydrogel (shell) generated in Example 22 are shown in Table 5 below. The cells were cultured together with the hydrogel in the liquid culture medium in the state of being encapsulated in the hydrogel in Example 22 as well. The conditions for cell culture are the same as those in Example 19. Through the above steps, a large number of cell aggregations were generated in the hydrogel.

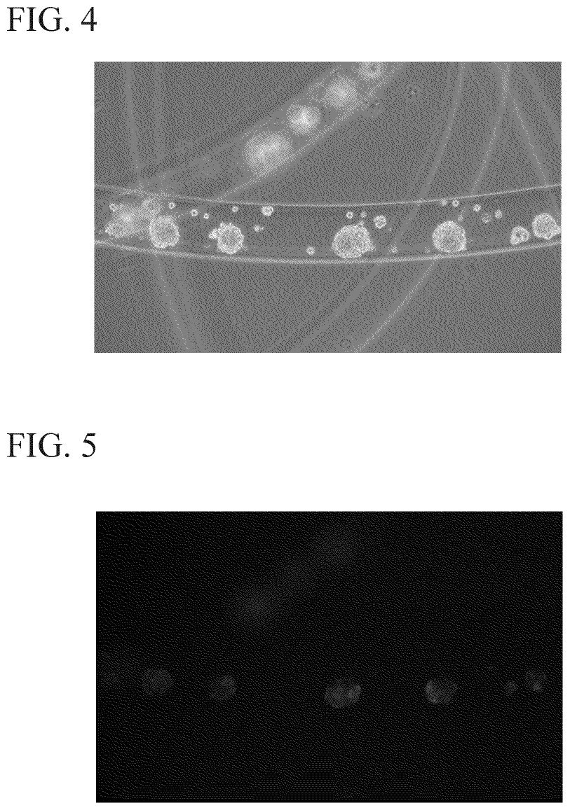

The cell population in Example 22 is constituted by the hydrogel and the large number of cell aggregations encapsulated in this hydrogel (see also an enlarged micrograph of FIG. 4). Here, it has been able to confirm by the same method as that in Example 19 that each of the cell aggregations in the cell population of Example 22 is the human adipose-derived mesenchymal stem cells and the human iPS cell-derived cardiomyocytes mixed with each other and aggregated together. Herein, FIG. 5 shows a state in which a portion shown in FIG. 4 is observed with a fluorescence microscope. In FIG. 5, as a result of mixture between the human iPS cell-derived cardiomyocytes that emit fluorescence and the human adipose-derived mesenchymal stem cells that do not emit fluorescence, a large number of cell aggregations that emit fluorescence in a mottled pattern are shown.

### Example 23

Next, a cell population according to Example 23 will be described. The cell population according to Example 23 was produced in the same manner as in Example 20 except for the initial cell density of the human iPS cell-derived cardiomyocytes. In Example 23, the initial cell density of the human iPS cell-derived cardiomyocytes was 5×10⁷ cells/mL.

The outer diameter and inner diameter of the tubular hydrogel (shell) generated in Example 23 are shown in Table 5 below. The cells were cultured together with the hydrogel in the liquid culture medium in the state of being encapsulated in the hydrogel in Example 23 as well. The conditions for cell culture are the same as those in Example 19. Through the above steps, a large number of cell aggregations were generated in the hydrogel.

### Example 24

Next, a cell population according to Example 24 will be described. The cell population according to Example 24 was produced in the same manner as in Example 21 except for the initial cell density of the human adipose-derived mesenchymal stem cells. In Example 24, the initial cell density of the human adipose-derived mesenchymal stem cells was 5×10⁷ cells/mL.

The outer diameter and inner diameter of the tubular hydrogel (shell) generated in Example 24 are shown in Table 5 below. The cells were cultured together with the hydrogel in the liquid culture medium in the state of being encapsulated in the hydrogel in Example 24 as well. The conditions for cell culture are the same as those in Example 19. Through the above steps, a large number of cell aggregations were generated in the hydrogel.

### Example 25

Next, a cell population according to Example 25 will be described. The cell population according to Example 25 was produced in the same manner as in Example 22 except for the culture conditions of the cells or cell aggregations encapsulated in the hydrogel. In Example 25, the cells or cell aggregations encapsulated in the hydrogel were cultured by shaking culture as dynamic culture. Through the above steps, a large number of cell aggregations were generated in the hydrogel.

The cell population in Example 25 is constituted by the hydrogel and the large number of cell aggregations encapsulated in this hydrogel. Here, it has been able to confirm by the same method as that in Example 19 that each of the cell aggregations in the cell population of Example 25 is the human adipose-derived mesenchymal stem cells and the human iPS cell-derived cardiomyocytes mixed with each other and aggregated together.

The mean value of the sizes of the large number of cell aggregations constituting the cell population in Examples 19 to 25 described above, the standard deviation thereof, and the coefficient of variation were calculated. The mean value of the sizes of the cell aggregations and the standard deviation thereof were calculated in the state of being encapsulated in the hydrogel by the methods described above. Herein, it should be noted that these numerical values are calculated from values measured for a large number of cell aggregations (samples) randomly extracted from the large number of cell aggregations constituting the cell population, in consideration of the above-described cut-off value. The calculation results are shown in Table 5 below.

TABLE 5

| | | | Outer | Inner | Initial | Mean Size | Standard Deviation | |
|---|---|---|---|---|---|---|---|---|
| | | | Diameter | Diameter | Cell | of Cell | of Sizes of Cell | Coefficient |
| | | Cultivation | of Shell | of Shell | Density | Aggregations | Aggregations | of Variation |
| | Cells | Method | (μm) | (μm) | (cells/mL) | μ (μm) | σ (μm) | σ/μ |
| Example 19 | Cardiomyocytes + | Static | Table 4 | 118 | 1 × 10⁷ | 77.9 | 16.8 | 0.22 |
| | Mesenchymal | culture | | | | | | |
| | stem cell | | | | | | | |
| Example 20 | Cardiomyocytes | Static | 235 | 120 | 1 × 10⁷ | 88.3 | 26.3 | 0.30 |
| | | culture | | | | | | |
| Example 21 | Mesenchymal | Static | 239 | 109 | 1 × 10⁷ | 88.4 | 20.0 | 0.23 |
| | stem cell | culture | | | | | | |
| Example 22 | Cardiomyocytes + | Static | 239 | 113 | 5 × 10⁷ | 113.2 | 25.3 | 0.22 |
| | Mesenchymal | culture | | | | | | |
| | stem cell | | | | | | | |
| Example 23 | Cardiomyocytes | Static | 244 | 124 | 5 × 10⁷ | 115.7 | 29.3 | 0.25 |
| | | culture | | | | | | |
| Example 24 | Mesenchymal | Static | 239 | 111 | 5 × 10⁷ | 120.2 | 25.4 | 0.21 |
| | stem cell | culture | | | | | | |
| Example 25 | Cardiomyocytes + | Dynamic | 238 | 110 | 5 × 10⁷ | 112.6 | 22.2 | 0.20 |
| | Mesenchymal | Culture | | | | | | |
| | stem cell | | | | | | | |

Table 5 shows that the coefficient of variation obtained by dividing the standard deviation of the sizes of the large number of cell aggregations constituting the cell population by the mean value of the sizes of the large number of cell aggregations in Examples 19 to 25 has a small value of 0.3 or less. Therefore, in Examples 19 to 25, the distribution of the sizes of the large number of cell aggregations constituting the cell population is sharp. In other words, in Examples 19 to 25, the sizes of the large number of cell aggregations constituting the cell population are uniformized.

Comparison between Example 19 and Examples 20 and 21 shows that a variation in the sizes of the large number of cell aggregations composed of the mixture of different types of cells is not significantly different from a variation in the sizes of the cell population including the large number of cell aggregations each composed of one type of cell. This can also be understood by comparing Example 22 and Examples 23 and 24. That is, by combining the conditions (the diameter of the hydrogel and the initial cell density) that can reduce the variation in the sizes of the large number of cell aggregations composed of one type of cell aggregation, it is possible to reduce the variation in the sizes of the large number of cell aggregations composed of the mixture of different types of cells.

In addition, comparison between Example 22 and Example 25 shows that in a case where the cell aggregations are formed in the state of being encapsulated in the hydrogel, there is no significant difference in the variation in the sizes of the large number of cell aggregations constituting the cell population in both static culture and dynamic culture.

In addition, the mean value of the aspect ratios of the large number of cell aggregations constituting the cell population in Examples 19 to 21 described above, the standard deviation thereof, and the coefficient of variation were calculated. The mean value of the aspect ratios of the cell aggregations and the standard deviation thereof were calculated in the state of being encapsulated in the hydrogel. The mean value of the aspect ratios of the cell aggregations and the standard deviation thereof were calculated by the methods described above. Herein, it should be noted that these numerical values are calculated from values measured for a large number of cell aggregations (samples) randomly extracted from the large number of cell aggregations constituting the cell population, in consideration of the above-described cut-off value. The calculated mean value of the aspect ratios of the cell aggregations and the standard deviation thereof are shown in Table 6 below. Herein, in each Example, the distribution of the aspect ratios of the large number of cell aggregations had a shape with a peak near the mean value of the aspect ratios.

TABLE 6

| | | | Mean Value of | Standard Deviation of | |
|---|---|---|---|---|---|
| | | Initial Cell | Aspect Ratios of | Aspect Ratios of Cell | Coefficient |
| | | Density | Cell Aggregations | Aggregations | of Variation |
| | Cells | (cells/mL) | μ | σ | σ/μ |
| Example 19 | Cardiomyocytes | 1 × 10⁷ | 1.17 | 0.13 | 0.11 |
| | + | | | | |
| | Mesenchymal | | | | |
| | stem cell | | | | |
| Example 20 | Cardiomyocytes | 1 × 10⁷ | 1.25 | 0.36 | 0.28 |
| Example 21 | Mesenchymal | 1 × 10⁷ | 1.30 | 0.24 | 0.18 |
| | stem cell | | | | |

Table 6 shows that the coefficient of variation obtained by dividing the standard deviation of the aspect ratios of the large number of cell aggregations constituting the cell population by the mean value of the aspect ratios of the large number of cell aggregations in Examples 19 to 21 has a small value of 0.3 or less. Therefore, in Examples 19 to 21, the distribution of the aspect ratios of the large number of cell aggregations constituting the cell population is sharp. In other words, in Examples 19 to 21, the sizes of the large number of cell aggregations constituting the cell population are uniformized.

Comparison between Example 19 and Examples 20 and 21 shows that mixing the human adipose-derived mesenchymal stem cells with the human iPS cell-derived cardiomyocytes makes the distribution of the aspect ratios of the large number of cell aggregations constituting the cell population sharper. The initial cell density of the human iPS cell-derived cardiomyocytes and the human adipose-derived mesenchymal stem cells is preferably lower as described above from the viewpoint of obtaining a sharper aspect ratio distribution.

### Example 26

Example 26 will be described in detail. The following reagents were prepared.
Normal human peripheral blood mononuclear cells (PBMC), frozen “Precision for Medicine”
Reagent for core: 0.3 w/v % methylcellulose solution (R&D scientific Inc.)
Alginic acid derivative: sodium alginate (“High-G ALG300” manufactured by KIMICA Corporation)
Culture medium: medium of KBM NK kit (Kohjin Bio Co., Ltd.)
(1) KBM NKCC-1 (medium for primary culture)
(2) KBM NKCC-2 (medium for expansion culture)

First, frozen normal human peripheral blood mononuclear cells (PBMC) were thawed, and precultured for 6 days in the above medium for primary culture according to the protocol of KBM NK kit (Kohjin Bio Co., Ltd.).

Subsequently, a cell suspension, an alginic acid derivative (hydrogel derivative), and a sheath solution were prepared. The sheath solution is an aqueous solution containing 100 mM calcium chloride and 3 w/v % sucrose.

The alginic acid derivative (hydrogel precursor) is a sodium alginate solution. The sodium alginate solution was generated by adding the above sodium alginate to a physiological saline solution and stirring the obtained solution. The concentration of the sodium alginate relative to the physiological saline solution was 0.99 mass percent concentration.

The cell suspension was obtained by introducing the above precultured PBMCs into a 0.6 w/v % methylcellulose solution (solvent: medium for expansion culture described above). The density (initial cell density) of the PBMCs in the cell suspension was 4×10⁶ cells/mL as shown in Table 7 below.

Using the above cell suspension, alginic acid derivative (hydrogel precursor), and sheath solution, a cell population was produced on the basis of the “Method 1 for producing cell population” described above (see also FIG. 3). That is, the flow of the cell suspension, the flow of the alginic acid derivative around the flow of the cell suspension, and the flow of the sheath solution around the flow of the alginic acid derivative were formed, and these flows were discharged into a physiological saline solution. The alginic acid derivative was crosslinked by contact with the aqueous calcium chloride solution as the sheath solution to form a tubular alginate gel.

As a result, a tubular hydrogel encapsulating the PBMCs was generated in the physiological saline solution. The outer diameter and inner diameter of the tubular hydrogel (shell) generated are shown in Table 7 below. The outer diameter and inner diameter of the shell shown in Table 7 are values immediately after formation of the shell.

The tubular hydrogel encapsulating the cells was left still in the physiological saline solution for a desired time. Subsequently, the tubular hydrogel encapsulating the cells was transferred to a liquid culture medium, and the cells started to be cultured in the tubular hydrogel.

The cells were cultured together with the hydrogel in the liquid culture medium (the above medium for expansion culture) in the state of being encapsulated in the hydrogel. The culture method was static culture. The cells were cultured at a temperature of 37° C. for 6 days. The culture medium was added so that the amount of culture medium was doubled on each of day 3 and day 6. Thus, on day 6, the amount of culture medium was 4 times the original amount.

The whole amount of culture medium was replaced on day 7 and day 8. On day 9, a large number of cell aggregations encapsulated in the hydrogel were photographed, and the mean value and standard deviation of the sizes of the large number of cell aggregations, and the coefficient of variation thereof were calculated.

### Example 27

A cell population according to Example 27 was produced in the same manner as in Example 26 except for the culture method of the cells and cell aggregations encapsulated in the hydrogel. Culture of the cells encapsulated in the hydrogel was performed in the same manner as in Example 26 until day 5.

In Example 27, the amount of culture medium was increased on day 6, and the cells and cell aggregations encapsulated in the hydrogel were then dynamically cultured (shake-cultured) at a speed of 125 rpm using a 125 mL Erlenmeyer flask. The whole amount of culture medium was replaced on day 7 and day 8. On day 9, a large number of cell aggregations encapsulated in the hydrogel were photographed, and the mean value and standard deviation of the sizes of the large number of cell aggregations, and the coefficient of variation thereof were calculated.

### Reference Example 3

A cell population according to Reference Example 3 was produced by subjecting the above frozen normal human peripheral blood mononuclear cells (PBMC) to suspension culture without being encapsulated in a hydrogel. First, frozen normal human peripheral blood mononuclear cells (PBMC) were thawed, and precultured for 6 days in the above medium for primary culture according to the protocol of KBM NK kit (Kohjin Bio Co., Ltd.). After the PBMCs were precultured, 5×10⁴ cells were suspended in 200 μL of the above medium for expansion culture. The PBMCs thereby started to be cultured (day 0).

The cell culture method was static culture. The cells were cultured at a temperature of 37° C. for 6 days. The culture medium was added so that the amount of culture medium was doubled on each of day 3 and day 6. Thus, on day 6, the amount of culture medium was 4 times the original amount. On day 8, the culture medium was added so that the amount of culture medium increased by 8 times.

On day 9, a large number of cell aggregations were photographed, and the mean value and standard deviation of the sizes of the large number of cell aggregations, and the coefficient of variation thereof were calculated.

The mean value and standard deviation of the sizes of the large number of cell aggregations constituting the cell population in Examples 26 and 27 and Reference Example 3, and the coefficient of variation thereof are shown in Table 7 below.

TABLE 7

| | | Outer | Inner | Initial | Mean Size | Standard Deviation | |
|---|---|---|---|---|---|---|---|
| | | Diameter | Diameter | Cell | of Cell | of Sizes of Cell | Coefficient |
| | | of Shell | of Shell | Density | Aggregations | Aggregations | of Variation |
| | Cultivation Method | (μm) | (μm) | (cells/mL) | μ (μm) | σ (μm) | σ/μ |
| Example 26 | Static Culture | 425 | 222 | 4 × 10⁶ | 198.1 | 24.3 | 0.12 |
| | (Entire Period) | | | | | | |
| Example 27 | Dynamic Culture | 413 | 216 | 4 × 10⁶ | 204.3 | 21.9 | 0.11 |
| | (after the 6th day) | | | | | | |
| Reference | Suspension Culture | — | — | — | 113.6 | 80.0 | 0.70 |
| Example 3 | | | | | | | |

In the above 26 and 27, it has been found that the PBMCs form a cell aggregation inside the tubular hydrogel despite being non-adherent cells. Furthermore, Table 7 shows that the coefficient of variation obtained by dividing the standard deviation of the sizes of the large number of cell aggregations constituting the cell population by the mean value of the sizes of the large number of cell aggregations in Examples 26 to 27 has a much smaller value than that in Reference Example 3. Therefore, in Examples 26 and 27, the distribution of the sizes of the large number of cell aggregations constituting the cell population is sharp. In other words, in Examples 26 and 27, the sizes of the large number of cell aggregations constituting the cell population are uniformized.

Furthermore, Example 27 shows that the cell aggregations composed of the PBMCs, which are non-adherent cells, do not collapse even by dynamic culture and maintain a uniform size as long as they are encapsulated in the tubular hydrogel.

### Example 28

Example 28 will be described in detail. The following reagents were prepared.
Human bone marrow-derived mesenchymal stem cells (PromoCell)
Reagent for core: 0.3 w/v % methylcellulose solution
Alginic acid derivative: sodium alginate (“High-G ALG300” manufactured by KIMICA Corporation)

First, human bone marrow-derived mesenchymal stem cells were cultured in a culture plate using a culture medium (MSC growth medium 2).

Subsequently, a cell suspension, an alginic acid derivative (hydrogel derivative), and a sheath solution were prepared. The sheath solution is an aqueous solution containing 100 mM calcium chloride and 3 w/v % sucrose.

The alginic acid derivative (hydrogel precursor) is a sodium alginate solution. The sodium alginate solution was generated by adding the above sodium alginate to a physiological saline solution and stirring the obtained solution. The concentration of the sodium alginate relative to the physiological saline solution was 1.44 mass percent concentration.

The cell suspension was obtained by introducing the human bone marrow-derived mesenchymal stem cells collected from the culture plate using a cell detachment enzyme (Accutase), into a 0.3 w/v % methylcellulose solution (solvent: MSC growth medium 2 described above). The density (initial cell density) of the human bone marrow-derived mesenchymal stem cells in the cell suspension was 5×10⁷ cells/mL.

Using the above cell suspension, alginic acid derivative (hydrogel precursor), and sheath solution, a cell population was produced on the basis of the “Method 1 for producing cell population” described above (see also FIG. 3). That is, the flow of the cell suspension, the flow of the alginic acid derivative around the flow of the cell suspension, and the flow of the sheath solution around the flow of the alginic acid derivative were formed, and these flows were discharged into a physiological saline solution. The alginic acid derivative was crosslinked by contact with the aqueous calcium chloride solution as the sheath solution to form a tubular alginate gel.

As a result, a tubular hydrogel encapsulating the human bone marrow-derived mesenchymal stem cells was generated in the physiological saline solution.

The tubular hydrogel encapsulating the cells was left still in the physiological saline solution for a desired time. Subsequently, the supernatant of the physiological saline solution was removed, the culture medium (MSC growth medium 2) was added, and the cells were cultured overnight in a 6 well plate at 37° C.

Through the above steps, a large number of cell aggregations were generated in the hydrogel. The cell population in Example 28 is constituted by the hydrogel and the large number of cell aggregations encapsulated in this hydrogel.

### Example 29

Next, a cell population according to Example 29 will be described. The cell population according to Example 29 was produced in the same manner as in Example 28 except for the concentration of the reagent for the core. In Example 29, the reagent for the core at the time of generating the tubular hydrogel was a 1.0 w/v % methylcellulose solution.

### Example 30

Next, a cell population according to Example 30 will be described. The cell population according to Example 30 was produced in the same manner as in Example 28 except for the concentration of the alginic acid derivative (hydrogel precursor). In Example 30, the concentration of the sodium alginate relative to the physiological saline solution at the time of generating the tubular hydrogel was 0.75 mass percent concentration.

### Example 31

Next, a cell population according to Example 31 will be described. The cell population according to Example 31 was produced in the same manner as in Example 28 except for the concentration of the reagent for the core and the concentration of the alginic acid derivative (hydrogel precursor). In Example 31, the reagent for the core at the time of generating the tubular hydrogel was a 1.0 w/v % methylcellulose solution. In addition, in Example 31, the concentration of the sodium alginate relative to the physiological saline solution at the time of generating the tubular hydrogel was 0.75 mass percent concentration.

Herein, the viscosity of the alginic acid derivative (hydrogel precursor) used at the time of producing the tubular hydrogel will be described. When the concentration of the sodium alginate relative to the physiological saline solution was 0.99 mass percent concentration, the viscosity of the alginic acid derivative used was 250 to 400 mPa·s at 20° C. Herein, it is known that the viscosity of the alginic acid derivative increases exponentially with respect to the concentration of the alginic acid derivative. Specifically, it is known that the viscosity of the alginic acid derivative increases approximately by 5 times when the concentration of the alginic acid derivative is doubled. Therefore, in Examples 28 and 29, the viscosity of the alginic acid derivative is estimated to be 550 to 950 mPa's at 20° C. Similarly, in Examples 30 and 31, the viscosity of the alginic acid derivative is estimated to be 110 to 190 mPa·s at 20° C.

The mean value of the sizes of the large number of cell aggregations constituting the cell population in Examples 28 to 31 described above, the standard deviation thereof, and the coefficient of variation were calculated. The mean value of the sizes of the cell aggregations and the standard deviation thereof were calculated in the state of being encapsulated in the hydrogel in Examples 28 to 31. The mean value of the sizes of the cell aggregations and the standard deviation thereof were calculated by the methods described above. Herein, it should be noted that these numerical values are calculated from values measured for a large number of cell aggregations (samples) randomly extracted from the large number of cell aggregations constituting the cell population, in consideration of the above-described cut-off value. These results are shown in Table 8 below. Herein, in each Example, the distribution of the sizes of the large number of cell aggregations had a shape with a peak near the mean value of the sizes.

TABLE 8

| | | | Viscosity of | | | | |
|---|---|---|---|---|---|---|---|
| | | | Alginic Acid | | | | |
| | | Concentration | Derivative | | | Standard | |
| | Concentration of | of Alginic | (Sodium | Initial | Mean Size | Deviation of | |
| | Methylcellulose | Acid in Shell | Alginate | Cell | of Cell | Sizes of Cell | Coefficient |
| | in Core | (percentage | Solution) | Density | Aggregations | Aggregations | of Variation |
| | (w/v %) | by mass) | (mPa · s) | (cells/mL) | μ (μm) | σ (μm) | σ/μ |
| Example 28 | 0.3 | 1.44 | 550~950 | 5 × 10⁷ | 207.7 | 50.7 | 0.24 |
| Example 29 | 1.0 | 1.44 | 550~950 | 5 × 10⁷ | 204.9 | 51.2 | 0.25 |
| Example 30 | 0.3 | 0.75 | 110~190 | 5 × 10⁷ | 183.5 | 62.1 | 0.34 |
| Example 31 | 1.0 | 0.75 | 110~190 | 5 × 10⁷ | 213.3 | 79.8 | 0.37 |

The methylcellulose of the core is known as a thickener. Thus, the higher the concentration of the methylcellulose of the core, the higher the viscosity of the core during production of the tubular hydrogel. Table 8 shows that the concentration of the methylcellulose of the core hardly affects the variation in the sizes of the cell aggregations, at least in the measured concentration range.

The higher the concentration of the alginic acid derivative (hydrogel precursor), the higher the viscosity of the shell during production of the tubular hydrogel and the higher the gel strength of the hydrogel produced. Table 8 shows that the variation in the sizes of the cell aggregations is reduced as the concentration of the alginic acid of the shell is higher.

Experimental results of differentiating the cell aggregations in Examples 28 to 31 will be described. First, the cell aggregations produced in Examples 28 to 31 were cultured in a differentiation induction medium in the state of being encapsulated in the hydrogel. Mesenchymal Stem Cell Chondrogenic Differentiation Medium (Ready-to-use) (Promo Cell/C-28012) was used as the differentiation induction medium. The differentiation induction medium was replaced every 3 days, and the human bone marrow-derived mesenchymal stem cells were cultured in the state of being encapsulated in the hydrogel for 24 days.

Through the above steps, a large number of cell aggregations were produced in the state of being encapsulated in the hydrogel. The mean value of the sizes of the large number of cell aggregations constituting the cell population in Examples 28 to 31, the standard deviation thereof, and the coefficient of variation were calculated. The mean value of the sizes of the cell aggregations and the standard deviation thereof were calculated in the state of being encapsulated in the hydrogel in Examples 28 to 31. The mean value of the sizes of the cell aggregations and the standard deviation thereof were calculated by the methods described above. Herein, it should be noted that these numerical values are calculated from values measured for a large number of cell aggregations (samples) randomly extracted from the large number of cell aggregations constituting the cell population, in consideration of the above-described cut-off value. These results are shown in Table 9 below.

In addition, the mean value of the aspect ratios of the large number of cell aggregations constituting the cell population in Examples 28 to 31 described above, the standard deviation thereof, and the coefficient of variation were calculated. The mean value of the aspect ratios of the cell aggregations and the standard deviation thereof were calculated in the state of being encapsulated in the hydrogel. The mean value of the aspect ratios of the cell aggregations and the standard deviation thereof were calculated by the methods described above. Herein, it should be noted that these numerical values are calculated from values measured for a large number of cell aggregations (samples) randomly extracted from the large number of cell aggregations constituting the cell population, in consideration of the above-described cut-off value. The calculated mean value of the aspect ratios of the cell aggregations and the standard deviation thereof are shown in Table 10 below.

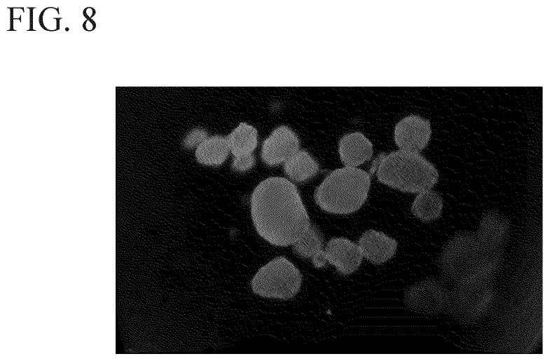

After the mesenchymal stem cells were cultured in the differentiation induction medium, the hydrogel was removed, and the cell aggregations were collected. With the collected cell aggregations stained with Alcian blue, it has been found that almost all the cell aggregations are fluorescent in blue (see FIG. 8). This shows that the mesenchymal stem cells are differentiated into chondrocytes.

With reference to Table 9 below, it can be seen that the coefficient of variation obtained by dividing the standard deviation of the sizes of the large number of cell aggregations constituting the cell population by the mean value of the sizes of the large number of cell aggregations in all Examples 28 to 31 is small. Therefore, the sizes of the large number of cell aggregations constituting the cell population are uniformized.

TABLE 9

| | | | Viscosity of | | | |
|---|---|---|---|---|---|---|
| | | | Alginic Acid | | | |
| | | Concentration | Derivative | | Standard | |
| | Concentration of | of Alginic | (Sodium | Mean Size | Deviation of | |
| | Methylcellulose | Acid in Shell | Alginate | of Cell | Sizes of Cell | Coefficient |
| | in Core | (percentage | Solution) | Aggregations | Aggregations | of Variation |
| | (w/v %) | by mass) | (mPa · s) | μ (μm) | σ (μm) | σ/μ |
| Example 28 | 0.3 | 1.44 | 550~950 | 108.5 | 32.7 | 0.30 |
| Example 29 | 1.0 | 1.44 | 550~950 | 107.0 | 31.2 | 0.29 |
| Example 30 | 0.3 | 0.75 | 110~190 | 94.9 | 31.0 | 0.33 |
| Example 31 | 1.0 | 0.75 | 110~190 | 103.4 | 41.2 | 0.40 |

With reference to Table 10 below, it can be seen that the coefficient of variation obtained by dividing the standard deviation of the aspect ratios of the shapes of the large number of cell aggregations constituting the cell population by the mean value of the aspect ratios of the shapes of the large number of cell aggregations in all Examples 28 to 31 is small. Therefore, the shapes of the large number of cell aggregations constituting the cell population are uniformized.

TABLE 10

| | | | Viscosity of | | | |
|---|---|---|---|---|---|---|
| | | | Alginic Acid | | | |
| | | Concentration | Derivative | | Standard | |
| | Concentration of | of Alginic | (Sodium | Mean Value of | Deviation of | |
| | Methylcellulose | Acid in Shell | Alginate | Aspect Ratios | Aspect Ratios | Coefficient |
| | in Core | (percentage | Solution) | of Cell | of Cell | of Variation |
| | (w/v %) | by mass) | (mPa · s) | Aggregations | Aggregations | σ/μ |
| Example 28 | 0.3 | 1.44 | 550~950 | 1.16 | 0.20 | 0.18 |
| Example 29 | 1.0 | 1.44 | 550~950 | 1.14 | 0.12 | 0.11 |
| Example 30 | 0.3 | 0.75 | 110~190 | 1.18 | 0.16 | 0.14 |
| Example 31 | 1.0 | 0.75 | 110~190 | 1.17 | 0.12 | 0.10 |

As described above, it is found that in a case where the sizes and/or aspect ratios of the mesenchymal stem cells before differentiation are uniformized, the sizes and/or aspect ratios of the mesenchymal stem cells after differentiation are also uniformized. Therefore, by differentiating the mesenchymal stem cells after reducing the variation in the sizes and/or aspect ratios of the mesenchymal stem cells in the state of being encapsulated in the hydrogel, the cell population including the differentiated cell aggregations having a uniform size and/or aspect ratio can be obtained.

In addition, since the variation in the sizes and/or aspect ratios of the mesenchymal stem cells before differentiation is reduced, the number of cell aggregations having an appropriate size and/or aspect ratio for differentiation increases, and thus the efficiency of differentiation can be improved.

In a case where stem cells such as the mesenchymal stem cells are differentiated in the state of being encapsulated in the hydrogel, it is not always necessary to reduce the variation in the sizes and/or aspect ratios of the stem cells before differentiation. The sizes and/or aspect ratios of the stem cells are controlled to some extent as long as they are encapsulated in the hydrogel. Therefore, it is considered that the efficiency of differentiation can be improved as described above. From such a viewpoint, it should be noted that the following invention can also be read explicitly from the disclosure of the present invention.

“A Method for Differentiating Cells Including:
encapsulating stem cells having multipotency or totipotency and/or progenitor cells having oligopotency or unipotency in a hydrogel; and
differentiating the stem cells and/or the progenitor cells in a state of being encapsulated in the hydrogel.”

In this case, the hydrogel preferably has the above-described shape. The type of the stem cells and/or the progenitor cells and the cell type after differentiation are also as described above. Preferably, a cell aggregation is formed in a state of being encapsulated in the hydrogel, and the cell aggregation is differentiated in the state of being encapsulated in the hydrogel. Differentiation can be performed by immersing the cells together with the hydrogel in a differentiation induction medium.

### Example 32

Next, a cell population according to Example 32 will be described. The cell population according to Example 32 was produced in the same manner as in Example 32 except for the concentration of the reagent for the core, the concentration of the alginic acid derivative (hydrogel precursor), and the number of days of culture. In Example 32, the reagent for the core at the time of generating the tubular hydrogel was a 0.1 w/v % methylcellulose solution. In addition, in Example 32, the concentration of the sodium alginate relative to the physiological saline solution at the time of generating the tubular hydrogel was 0.75 mass percent concentration.

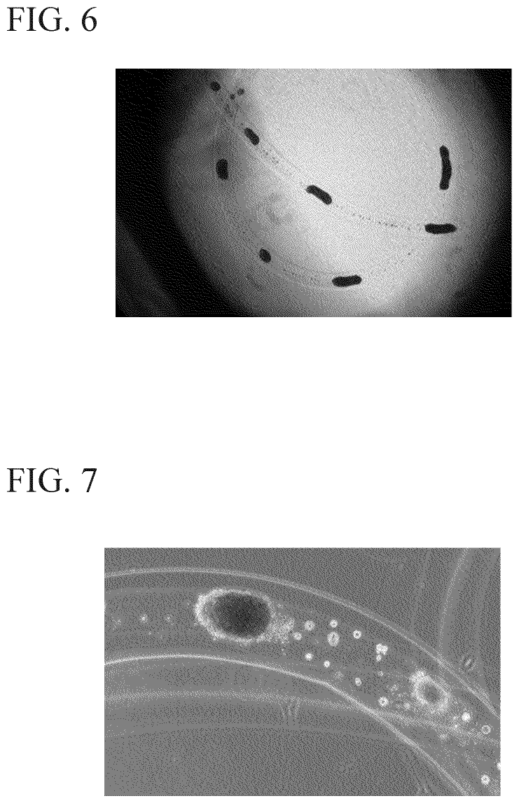

In Example 32, the cells were cultured in the tubular hydrogel for 7 days. FIG. 6 is a micrograph of the cell aggregations constituting the cell population in Example 32 on day 1 of culture. FIG. 7 is a micrograph of the cell aggregations constituting the cell population in Example 32 on day 7 of culture.

From FIGS. 6 and 7, it can be seen that the shapes of the cell aggregations approach a spherical shape as the days of culture progress. Therefore, the aspect ratios of the shapes of the cell aggregations approach “1” as the days of culture progress. As a result, it is predicted that the aspect ratios of the shapes of the cell aggregations also decrease. Therefore, it is considered that the variation in the aspect ratios of the cell aggregations can be reduced by controlling the number of days of culture.

As described above, the contents of the present invention have been disclosed through the embodiments and examples, but it should not be understood that the description and the drawings constituting a part of the disclosure limit the present invention. From this disclosure, various alternative embodiments, examples, and operational techniques will become apparent to those skilled in the art. Therefore, the technical scope of the present invention is defined only by the matters specifying the invention according to the claims appropriate from the above description.

1. A cell population comprising:
a core comprising a cell suspension including a large number of cell aggregations; and
a hydrogel surrounding the core, wherein
when a cell and a cell aggregation having a size less than a predetermined cut-off value is removed,
a coefficient of variation obtained by dividing a standard deviation of aspect ratios of shapes of the large number of cell aggregations by a mean value of the aspect ratios of the shapes of the large number of cell aggregations is 0.30 or less.

2. The cell population according to claim 1, comprising:
a tubular shell formed by the hydrogel.

3. The cell population according to claim 1, wherein the hydrogel has a capsule shape which is capable of dividing the large number of cell aggregations into a plurality of cell aggregations.

4. The cell population according to claim 1, wherein a storage modulus of the hydrogel in a state of encapsulating the large number of cell aggregations is 100 Pa or more.

5. The cell population according to claim 1, wherein
the hydrogel is an alginate gel, and
an M/G ratio of an alginic acid derivative for forming the alginate gel is 1.0 or less.

6. The cell population according to claim 1, wherein
the hydrogel is an alginate gel, and
an M/G ratio of an alginic acid derivative for forming the alginate gel is 0.1 or more.

7. The cell population according to claim 5, wherein the alginate gel is formed by gelling a solution in which a weight of the alginic acid derivative relative to a weight of a solvent containing the alginic acid derivative is between 0.5 wt % and 7.0 wt % inclusive.

8. The cell population according to claim 1, wherein a viscosity of a hydrogel derivative for forming the hydrogel is between 50 mPa and 10,000 mPa inclusive at a temperature of 20° C.

9. A cell population comprising
a large number of cell aggregations collected from inside the hydrogel which is surrounding the core comprising the cell suspension, wherein
when a cell and a cell aggregation having a size less than a predetermined cut-off value is removed,
a coefficient of variation obtained by dividing a standard deviation of aspect ratios of shapes of the large number of cell aggregations by a mean value of the aspect ratios of the shapes of the large number of cell aggregations is 0.30 or less.

10. The cell population according to claim 1, wherein the mean value of the aspect ratios of the shapes of the large number of cell aggregations is 1.5 or less when a cell and a cell aggregation having a size less than a predetermined cut-off value is removed.

11. The cell population according to claim 1, wherein the large number of cell aggregations contain adherent cells or non-adherent cells.

12. The cell population according to claim 1, wherein the large number of cell aggregations contain adherent cells that do not substantially proliferate in suspension.

13. The cell population according to claim 1, wherein each of the large number of cell aggregations contain different types of cells from each other.

14. The cell population according to claim 1, wherein the cell aggregations contain stem cell having multipotency or totipotency, progenitor cell having oligopotency or unipotency, or cell differentiated from stem cells or progenitor cells.

15. The cell population according to claim 1, wherein the cell aggregation contains cells differentiated from stem cells or progenitor cells in a state of being encapsulated in the hydrogel.

16. (canceled)

17. A method for producing a cell population comprising:
forming a structure comprising a core including a cell suspension and a shell formed by a hydrogel surrounding the core; and
culturing the cells in the shell so that, after removing a cell and a cell aggregation having a size less than a predetermined cut-off value, a coefficient of variation obtained by dividing a standard deviation of aspect ratios of shapes of a large number of cell aggregations by a mean value of the aspect ratios of the shapes of the large number of cell aggregations is 0.30 or less.

18. A method for producing a cell population comprising:
forming a structure comprising a core including a cell suspension and a shell formed by a hydrogel surrounding the core;
culturing the cells into the shell to form a large number of cell aggregations; and
collecting the large number of cell aggregations from inside the shell to obtain a cell population in which after removing a cell and a cell aggregation having a size less than a predetermined cut-off value, a coefficient of variation obtained by dividing a standard deviation of aspect ratios of shapes of a large number of cell aggregations by a mean value of the aspect ratios of the shapes of the large number of cell aggregations is 0.30 or less.

19. The method for producing a cell population according to claim 18, wherein the large number of cell aggregations are collected from inside the shell by dissolving the hydrogel solution using a solution containing EDTA or an alginate lyase.

20. The method for producing a cell population according to claim 17, wherein
the cell aggregation contain stem cell having multipotency or totipotency, and/or progenitor cell having oligopotency or unipotency,
the method comprising differentiating the stem cells and/or the progenitor cells in a state of being encapsulated in the hydrogel.

21. A biological substance generated by the large number of cell aggregations constituting the cell population according to claim 1.
